(19) United States
(12) Patent Application Publication
(10) Pub. No.: US 2017/0042775 A1
(43) Pub. Date: Feb. 16, 2017

(54) COSMETIC COMPOSITION

(71) Applicant: L'OREAL, Paris (FR)

(72) Inventor: Maki YAMAGUCHI, Kawasaki-shi, Kanagawa (JP)

(21) Appl. No.: 15/305,439

(22) PCT Filed: Apr. 15, 2015
(86) PCT No.: PCT/JP2015/062161

§ 371 Date: Oct. 20, 2016

(30) Foreign Application Priority Data
Apr. 24, 2014 (JP) 2014-089966

(51) Int. Cl. A61K 8/11; A61Q 5/12; A61Q 17/04; A61Q 5/02; A61Q 13/00; A61K 8/44; A61K 8/365; A61K 8/368; A61K 8/81; A61K 8/84; A61K 8/73; A61K 8/46

(52) U.S. Cl. CPC A61K 8/11; A61K 8/731; A61Q 5/12; A61Q 17/04; A61Q 5/02; A61K 8/463; A61K 8/442; A61K 8/365; A61K 8/368; A61K 8/8182; A61K 8/84; A61Q 13/00; A61K 2800/594; A61K 2800/5922; A61K 2800/5426; A61K 2800/412

(57) ABSTRACT

The present invention relates to a cosmetic composition comprising: (a) at least one anionic surfactant; (b) at least one hydrophobically-modified cationic polymer; (c) at least one water-insoluble particle comprising at least an inorganic part and/or a polymeric part and/or a visible light absorbing part; and (d) at least 40% by weight of water. The amount of the (b) hydrophobically-modified cationic polymer is preferably 0.005% by weight or more, more preferably 0.01% by weight or more, and even more preferably 0.05% by weight or more, relative to the total weight of the composition. The cosmetic composition according to the present invention can effectively deposit water-insoluble particles on the surface of various substrates such as skin and hair, and can deliver the benefits derived from the particles to the surface.

### TECHNICAL FIELD

The present invention relates to a cosmetic composition which can effectively deposit water-insoluble particles on substrates and can deliver aesthetic effects derived from the particles to the substrates, as well as a cosmetic process using the same.

### BACKGROUND ART

Some water-insoluble particles are known for their use as benefit-delivering agents in a variety of personal care compositions to deliver the benefits to the surface of various substrates such as skin and hair, if the particles are deposited on the surface. Examples of such benefit agents are oil absorbers such as microcapsules or microspheres, pigments or coloring agents, opacifiers, pearlescent agents, feel modifiers, skin protectants, matting agents, friction enhancers, slip agents, conditioning agents, exfoliants, odor absorbers, and cleaning enhancers. Additionally, many active ingredients are typically used in solid particulate form including antiperspirant agents, antidandruff agents, antimicrobials, antibiotics, and sunscreens.

Given a wide range of benefits delivered by water-insoluble particles, it is also desirable to have a rinse-off composition capable of depositing the particles on the surface of various substrates. However, since a large portion of particles is washed away during the rinsing process, the efficient deposition and retention of the particles on the surface are particularly difficult to obtain for compositions intended for cleansing the surface of the substrates.

To improve the deposition of water-insoluble particles on the surface of substrates, there have been attempts to use an excessive amount of particles, treat the surface of the particle properly with a surface modifier or modifiers, and the like as disclosed in WO 93/07862, JP-A-2000-143483, WO 01/26635, JP-B-3898060, JP-B-4447366 and US-A-2012/0145172. The degree of deposition of the particles on the surface, however, has not been enough to provide the desired benefits.

### DISCLOSURE OF INVENTION

An objective of the present invention is to provide a cosmetic composition which can effectively deposit water-insoluble particles on the surface of various substrates such as skin and hair, and can deliver the benefits derived from the particles to the surface.

The above objective can be achieved by a cosmetic composition comprising:
(a) at least one anionic surfactant;
(b) at least one hydrophobically-modified cationic polymer;
(c) at least one water-insoluble particle comprising at least an inorganic part and/or a polymeric part and/or a visible light absorbing part; and
(d) at least 40% by weight of water.

The amount of the (b) hydrophobically-modified cationic polymer may be 0.005% by weight or more, preferably 0.01% by weight or more, and more preferably 0.05% by weight or more, relative to the total weight of the composition. For example, the amount of the (b) hydrophobically-modified cationic polymer may range from 0.005% to 10% by weight, preferably from 0.01% to 5% by weight, and more preferably from 0.05% to 1% by weight, relative to the total weight of the composition.

The amount of the (a) anionic surfactant may range from 1 to 30% by weight, preferably from 3 to 25% by weight, and more preferably from 5 to 20% by weight, relative to the total weight of the composition.

The (b) hydrophobically-modified cationic polymer may be selected from the group consisting of
(1) cationic associative amphiphilic polyurethanes,
(2) quaternized cellulose derivatives,
(3) cationic poly(vinyllactam) polymers, and
(4) cationic polymer(s) obtained by polymerization of a monomer mixture comprising one or more vinyl monomers substituted with one or more amino groups, one or more hydrophobic nonionic vinyl monomers, and one or more associative vinyl monomers.

It is preferable that the (b) hydrophobically-modified cationic polymer comprise at least one quaternary ammonium group. It is more preferable that the quaternary ammonium group include at least one C₈-C₃₀ hydrocarbon group. It is preferable that the (b) hydrophobically-modified cationic polymer is a cellulose compound. The (b) hydrophobically-modified cationic polymer may be selected from hydrophobically-modified cationic cellulose polymers comprising at least one quaternary ammonium group including at least one C₈-C₃₀ hydrocarbon group.

The (c) water-insoluble particle may be in the form of a microcapsule or a microsphere.

The (c) water-insoluble particle may comprise at least one inorganic material, preferably selected from the group consisting of talc, mica, silica, kaolin, sericite, calcinated talc, calcinated mica, calcinated sericite, synthetic mica, bismuth oxychloride, barium sulfate, boron nitride, calcium carbonate, magnesium carbonate, magnesium hydrogen carbonate, and hydroxyapatite.

The (c) water-insoluble particle may comprise at least one organic material selected from the group consisting of polyurea, melamine-formaldehyde condensate, urea-formaldehyde condensate, aminoplasts, polyurethane, polyacrylate, polyphosphate, polystyrene, polyester, polyamide, polyolefin, polysaccharide, silicone, silicone resin, protein, modified cellulose, and gum.

The (c) water-insoluble particle of the present invention may include at least one additional cosmetic active agent, preferably hydrophobic and/or lipophilic cosmetic active agent, and more preferably a fragrance, a conditioning agent or a UV filter, in particular when the particle is a porous particle or a hollow particle.

The amount of the (c) water-insoluble particle may range from 0.01 to 10% by weight, preferably from 0.05 to 5% by weight, and more preferably from 0.1 to 3% by weight, relative to the total weight of the composition.

The (a) anionic surfactant may be selected from the group consisting of:

sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C14-16 olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, disodium cocoyl glutamate, potassium myristoyl glutamate, TEA-cocoyl glutamate, sodium cocoyl glycinate, potassium cocoyl glycinate, sodium cocoyl alaniate, TEA-cocoyl alaninate and mixtures thereof.

The cosmetic composition according to the present invention may further comprise at least one amphoteric or cationic surfactant.

The amount of the amphoteric or cationic surfactant may range from 0.01 to 30% by weight, preferably from 0.05 to 20% by weight, and more preferably from 0.1 to 10% by weight, relative to the total weight of the composition.

The amount of (d) water may range from 40 to 98% by weight or less, preferably from 45 to 95% by weight, and more preferably from 50 to 90% by weight relative to the total weight of the composition.

It is preferable that the cosmetic composition according to the present invention be intended for cleansing a keratin substance, more preferably keratin fibers, and even more preferably human hair.

The present invention also relates to a process for cleansing a keratin substance, preferably keratin fibers, and more preferably human hair, wherein the cosmetic composition according to the present invention is applied to the keratin substance, preferably keratin fibers, and more preferably human hair.

### BEST MODE FOR CARRYING OUT THE INVENTION

After diligent research, the inventor has discovered a cosmetic composition that can effectively deposit water-insoluble particles on the surface of various substrates such as skin and hair, and can deliver the benefits derived from the particles to the surface.

It has now been discovered that a hydrophobically-modified cationic polymer or polymers can surprisingly improve the deposition and retention of water-insoluble particles on the surface of various substrates, even when the hydrophobically-modified cationic polymer or polymers are used in a cosmetic composition including an anionic surfactant or surfactants.

Thus, the cosmetic composition according to the present invention comprises:
(a) at least one anionic surfactant;
(b) at least one hydrophobically-modified cationic polymer,
(c) at least one water-insoluble particle comprising at least an inorganic part and/or a polymeric part and/or a visible light absorbing part, and
(d) at least 40% by weight of water.

In the present invention, various types of water-insoluble particles can be applied.

Preferably, the water-insoluble particles are selected from microcapsules with polymeric walls containing at least one benefit-delivering agent such as oil/fragrance. The benefit-delivering agent can be delivered by the breaking of the microcapsule upon exposure to triggers such as friction, pH, moisture, or temperature, depending on the type of the microcapsule. The benefit-delivering agent can also be delivered over time from the microsphere through, for example, the crosslinked network of the microsphere. Accordingly, for example, the present invention can enhance the delivery of fragrance material(s) to the surface of skin or hair, and therefore, the skin or hair can enjoy benefits caused by the fragrance material(s) such as stronger fragrance effects at desired times and prolonged fragrance effects.

Hereafter, each of the phases constituting the cosmetic composition according to the present invention will be described in a detailed manner.

[Anionic Surfactant]

The cosmetic composition according to the present invention comprises at least one anionic surfactant, and two or more anionic surfactants may be used in combination. Thus, a single type of anionic surfactant or a combination of different types of anionic surfactants may be used.

The anionic surfactants may be chosen in particular from anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl polyglucosides, and their mixtures.

1) Anionic derivatives of proteins of vegetable origin are protein hydrolysates comprising a hydrophobic group, it being possible for the said hydrophobic group to be naturally present in the protein or to be added by reaction of the protein and/or of the protein hydrolysate with a hydrophobic compound. The proteins are of vegetable origin or derived from silk, and the hydrophobic group can in particular be a fatty chain, for example an alkyl chain comprising from 10 to 22 carbon atoms. Mention may more particularly be made, as anionic derivatives of proteins of vegetable origin, of apple, wheat, soybean or oat protein hydrolysates comprising an alkyl chain having from 10 to 22 carbon atoms, and their salts. The alkyl chain can in particular be a lauryl chain and the salt can be a sodium, potassium and/or ammonium salt.

Thus, mention may be made, as protein hydrolysates comprising a hydrophobic group, for example, of salts of protein hydrolysates where the protein is a silk protein modified by lauric acid, such as the product sold under the name Kawa Silk by Kawaken; salts of protein hydrolysates where the protein is a wheat protein modified by lauric acid, such as the potassium salt sold under the name Aminofoam W OR by Croda (CTFA name: potassium lauroyl wheat amino acids) and the sodium salt sold under the name Proteol LW 30 by Seppic (CTFA name: sodium lauroyl wheat amino acids); salts of protein hydrolysates where the protein is an oat protein comprising an alkyl chain having from 10 to 22 carbon atoms and more especially salts of protein hydrolysates where the protein is an oat protein modified by lauric acid, such as the sodium salt sold under the name Proteol OAT (30% aqueous solution) by Seppic (CTFA name: sodium lauroyl oat amino acids); or salts of apple protein hydrolysates comprising an alkyl chain having from 10 to 22 carbon atoms, such as the sodium salt sold under the name Proteol APL (30% aqueous/glycol solution) by Seppic (CTFA name: sodium cocoyl apple amino acids). Mention may also be made of the mixture of lauroyl amino acids (aspartic acid, glutamic acid, glycine, alanine) neutralized with sodium N-methylglycinate sold under the name Proteol SAV 50 S by Seppic (CTFA name: sodium cocoyl amino acids).

2) Mention may be made, as phosphates and alkyl phosphates, for example, of monoalkyl phosphates and dialkyl phosphates, such as lauryl monophosphate, sold under the name MAP 20® by Kao Chemicals, the potassium salt of dodecyl phosphate, the mixture of mono- and diesters (predominantly diester) sold under the name Crafol AP-31® by Cognis, the mixture of octyl phosphate monoester and diester, sold under the name Crafol AP-20® by Cognis, the mixture of ethoxylated (7 mol of EO) 2-butyloctyl phosphate monoester and diester, sold under the name Isofol 12 7 EO-Phosphate Ester® by Condea, the potassium or triethanolamine salt of mono(C₁₂-C₁₃)alkyl phosphate, sold under the references Arlatone MAP 230K-40® and Arlatone MAP 2301-60® by Uniqema, potassium lauryl phosphate, sold under the name Dermalcare MAP XC-99/09® by Rhodia Chimie, and potassium cetyl phosphate, sold under the name Arlatone MAP 160K by Uniqema.

3) Mention may be made, as carboxylates, of:
amido ether carboxylates (AEC), such as sodium lauryl amido ether carboxylate (3 EO), sold under the name Akypo Foam 30® by Kao Chemicals;
polyoxyethylenated carboxylic acid salts, such as oxyethylenated (6 EO) sodium lauryl ether carboxylate (65/25/10 C₁₂-C₁₄-C₁₆), sold under the name Akypo Soft 45 NV® by Kao Chemicals, polyoxyethylenated and carboxymethylated fatty acids originating from olive oil, sold under the name Olivem 400® by Biologia E Tecnologia, or oxyethylenated (6 EO) sodium tridecyl ether carboxylate, sold under the name Nikkol ECTD-6NEX® by Nikkol; and
salts of fatty acids (soaps) having a C₆ to C₂₂ alkyl chain which are neutralized with an organic or inorganic base, such as potassium hydroxide, sodium hydroxide, triethanolamine, N-methylglucamine, lysine and arginine.

4) Mention may in particular be made, as amino acid derivatives, of alkali salts of amino acids, such as:
sarcosinates, such as sodium lauroyl sarcosinate, sold under the name Sarkosyl NL 97® by Ciba or sold under the name Oramix L 30® by Seppic, sodium myristoyl sarcosinate, sold under the name Nikkol Sarcosinate TAN® by Nikkol, or sodium palmitoyl sarcosinate, sold under the name Nikkol Sarcosinate PN® by Nikkol;
alaninates, such as sodium N-lauroyl-N-methylamidopropionate, sold under the name Sodium Nikkol Alaninate LN 30® by Nikkol or sold under the name Alanone ALE® by Kawaken, or triethanolamine N-lauroyl-N-methylalanine, sold under the name Alanone ALTA® by Kawaken;
glutamates, such as triethanolamine monococoyl glutamate, sold under the name Acylglutamate CT-12® by Ajinomoto, triethanolamine lauroyl glutamate, sold under the name Acylglutamate LT-12® by Ajinomoto;
aspartates, such as the mixture of triethanolamine N-lauroyl aspartate and triethanolamine N-myristoyl aspartate, sold under the name Asparack® by Mitsubishi;
glycine derivatives (glycinates), such as sodium N-cocoyl glycinate, sold under the names Amilite GCS-12® and Amilite GCK 12 by Ajinomoto;
citrates, such as the citric monoester of oxyethylenated (9 mol) coco alcohols, sold under the name Witconol EC 1129 by Goldschmidt; and
galacturonates, such as sodium dodecyl D-galactoside uronate, sold by Soliance.

5) Mention may be made, as sulphosuccinates, for example, of oxyethylenated (3 EO) lauryl (70/30 C₁₂/C₁₄) alcohol monosulphosuccinate, sold under the names Setacin 103 Special® and Rewopol SB-FA 30 K 4® by Witco, the disodium salt of a hemisulphosuccinate of C₁₂-C₁₄ alcohols, sold under the name Setacin F Special Paste® by Zschimmer Schwarz, oxyethylenated (2 EO) disodium oleamidosulphosuccinate, sold under the name Standapol SH 135® by Cognis, oxyethylenated (5 EO) lauramide monosulphosuccinate, sold under the name Lebon A-5000® by Sanyo, the disodium salt of oxyethylenated (10 EO) lauryl citrate monosulphosuccinate, sold under the name Rewopol SB CS 50® by Witco, or ricinoleic monoethanolamide monosulphosuccinate, sold under the name Rewoderm S 1333® by Witco. Use may also be made of polydimethylsiloxane sulphosuccinates, such as disodium PEG-12 dimethicone sulphosuccinate, sold under the name Mackanate-DC 30 by MacIntyre.

6) Mention may be made, as alkyl sulphates, for example, of triethanolamine lauryl sulphate (CTFA name: TEA lauryl sulphate), such as the product sold by Huntsman under the name Empicol TL40 FL or the product sold by Cognis under the name Texapon T42, which products are at 40% in aqueous solution. Mention may also be made of ammonium lauryl sulphate (CTFA name: ammonium lauryl sulphate), such as the product sold by Huntsman under the name Empicol AL 30FL, which is at 30% in aqueous solution.

7) Mention may be made, as alkyl ether sulphates, for example, of sodium lauryl ether sulphate (CTFA name: sodium laureth sulphate), such as that sold under the names Texapon N40 and Texapon AOS 225 UP by Cognis, or ammonium lauryl ether sulphate (CTFA name: ammonium laureth sulphate), such as that sold under the name Standapol EA-2 by Cognis.

8) Mention may be made, as sulphonates, for example, of α-olefinsulphonates, such as sodium α-olefinsulphonate (C₁₄-C₁₆), sold under the name Bio-Terge AS-40® by Stepan, sold under the names Witconate AOS Protégé® and Sulframine AOS PH 12® by Witco or sold under the name Bio-Terge AS-40 CG® by Stepan, secondary sodium olefinsulphonate, sold under the name Hostapur SAS 30® by Clariant; or linear alkylarylsulphonates, such as sodium xylenesulphonate, sold under the names Manrosol SXS30®, Manrosol SXS400 and Manrosol SXS930 by Manro.

9) Mention may be made, as isethionates, of acylisethionates, such as sodium cocoylisethionate, such as the product sold under the name Jordapon CI P® by Jordan.

10) Mention may be made, as taurates, of the sodium salt of palm kernel oil methyltaurate, sold under the name Hostapon CT Pate® by Clariant; N-acyl-N-methyltaurates, such as sodium N-cocoyl-N-methyltaurate, sold under the name Hostapon LT-SF® by Clariant or sold under the name Nikkol CMT-30-T® by Nikkol, Sodium Methyl Stearoyl Taurate sold under the name Nikkol SMT® or sodium palmitoyl methyltaurate, sold under the name Nikkol PMT® by Nikkol.

11) The anionic derivatives of alkyl polyglucosides can in particular be citrates, tartrates, sulphosuccinates, carbonates and glycerol ethers obtained from alkyl polyglucosides. Mention may be made, for example, of the sodium salt of cocoylpolyglucoside (1,4) tartaric ester, sold under the name Eucarol AGE-ET® by Cesalpinia, the disodium salt of cocoylpolyglucoside (1,4) sulphosuccinic ester, sold under the name Essai 512 MP® by Seppic, or the sodium salt of cocoylpolyglucoside (1,4) citric ester, sold under the name Eucarol AGE-EC® by Cesalpinia.

For the amino acid derivatives, it is preferable that they are chosen from acyl glycine derivatives or glycine derivatives, in particular acyl glycine salt.

The acyl glycine derivatives or glycine derivatives can be chosen from acyl glycine salts (or acyl glycinates) or glycine salts (or glycinates), and in particular from the following.

i) Acyl glycinates of formula (I):

R—HNCH₂COOX (I)

in which
R represents an acyl group R′C═O, with R″, which represents a saturated or unsaturated, linear or branched, hydrocarbon chain, preferably comprising from 10 to 30 carbon atoms, more preferably from 12 to 22 carbon atoms, even more preferably from 14 to 22 carbon atoms and better still from 16 to 20 carbon atoms, and
X represents a cation chosen, for example, from the ions of alkali metals, such as Na, Li or K, preferably Na or K, the ions of alkaline earth metals, such as Mg, ammonium groups and their mixtures.

The acyl group can in particular be chosen from the lauroyl, myristoyl, behenoyl, palmitoyl, stearoyl, isostearoyl, olivoyl, cocoyl or oleoyl groups and their mixtures.

Preferably, R is a cocoyl group.

ii) Glycinates of following formula (II):

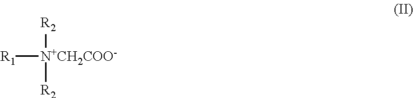

in which:
R₁ represents a saturated or unsaturated, linear or branched, hydrocarbon chain comprising from 10 to 30 carbon atoms, preferably from 12 to 22 carbon atoms and better still from 16 to 20 carbon atoms; R₁ is advantageously chosen from the lauryl, myristyl, palmityl, stearyl, cetyl, cetearyl or oleyl groups and their mixtures and preferably from the stearyl and oleyl groups,
the R₂ groups, which are identical or different, represent an R″OH group, R″ being an alkyl group comprising from 2 to 10 carbon atoms, preferably from 2 to 5 carbon atoms.

Mention may be made, as the compound of formula (I), for example, of the compounds carrying the INCI name sodium cocoyl glycinate, such as, for example, Amilite GCS-12, sold by Ajinomoto, or potassium cocoyl glycinate, such as, for example, Amilite GCK-12 from Ajinomoto.

Use may be made, as compounds of formula (II), of dihydroxyethyl oleyl glycinate or dihydroxyethyl stearyl glycinate.

Preferably anionic surfactants are not soaps. So preferably anionic surfactants are chosen from synthetic anionic surfactants. More preferably, anionic surfactants are chosen from amido ether carboxylates; alkyl sulfates; alkyl ether sulfates; olefin sulfonates and acylisethionates; and mixtures thereof.

It is preferable that the (a) anionic surfactant be selected from the group consisting of: sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C14-16 olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, disodium cocoyl glutamate, potassium myristoyl glutamate, TEA-cocoyl glutamate, sodium cocoyl glycinate, potassium cocoyl glycinate, sodium cocoyl alaniate, TEA-cocoyl alaninate and mixtures thereof.

The amount of the (a) anionic surfactant may range from 1 to 30% by weight, preferably from 3 to 25% by weight, and more preferably from 5 to 20% by weight, relative to the total weight of the composition.

[Hydrophobically-Modified Cationic Polymer]

The cosmetic composition according to the present invention comprises at least one hydrophobically-modified cationic polymer, and two or more hydrophobically-modified cationic polymers may be used in combination. Thus, a single type of hydrophobically-modified cationic polymer or a combination of different types of hydrophobically-modified cationic polymers may be used.

By “hydrophobically-modified cationic polymer”, it is meant a cationic polymer that contains at least one hydrophobic moiety, that could be present in the cationic polymer or that could be added by any way to a previously made cationic polymer.

The hydrophobically-modified cationic polymer can for example be prepared by hydrophobicizing a cationic polymer by, for example, substituting a hydrogen atom, a hydrophilic moiety or a short hydrocarbon chain such as a C₁-C₄ alkyl group of the cationic polymer with a hydrophobic moiety which includes typically a long chain hydrocarbon group, such as a C₈-C₃₀ alkyl, alkenyl, alkynyl, or aralkyl group, and a C₈-C₃₀ aryl group. It is preferable that the hydrophobic moiety is bonded to the polymer backbone of the cationic polymer via an ether group.

Preferably, said hydrophobic moiety is a C₈-C₃₀ hydrocarbon group, such as a C₈-C₃₀ alkyl group, linear or branched, saturated or unsaturated; preferably, it is a C₃-C₃₀, most preferably C₁₀-C₂₈ saturated and linear alkyl group.

Preferably, the hydrophobically-modified cationic polymer comprises at least one quaternary ammonium group. More preferably, the quaternary ammonium group includes at least one C₈-C₃₀ hydrocarbon group.

As used herein, the term “cationic polymer” is intended to mean any polymer containing at least one cationic group and/or at least one group which can be ionized to a cationic group. The term “polymer” here means a molecule having repeating units which preferably have a molecular weight of more than 5000, in particular more than 10000.

The cationic polymer to be used for the present invention may or may not have a siloxane moiety.

The cationic polymers which can be used in accordance with the present invention may be chosen from all those known in the art to improve the cosmetic properties of hair treated with detergent compositions, for example, those described in European Patent Application No. 0 337 354 and French Patent Application Nos. 2 270 846, 2 383 660, 2 598 611, 2 470 596, and 2 519 863.

In one embodiment, the cationic polymers are chosen, for example, from those which comprise units comprising at least one group chosen from primary, secondary, tertiary, and quaternary amine groups which can either be part of the main polymer chain, or can be carried by a side substituent directly connected to the chain.

The cationic polymers may have a weight-average molecular mass of greater than 10⁵, for example, greater than 10⁶, or ranging from 10⁶ to 10⁸.

The hydrophobically-modified cationic polymer according to the present invention may be a cationic associative polymer; and said cationic associative polymers may be cationic water-soluble or water-dispersible polymers that are capable, in an aqueous medium, of reversibly combining with each other or with other molecules.

In one embodiment, the cationic associative polymer used in the present invention is a cationic amphiphilic polymer, which means a polymer comprising at least one hydrophilic moiety which renders the polymer soluble in water and at least one hydrophobic moiety region, comprising at least one fatty chain, by means of which the polymer interacts and undergoes assembly with another associative polymer or with other molecules.

Hence the cationic associative polymer used in the present invention is preferably a cationic amphiphilic polymer which comprises at least one hydrophilic group and at least one fatty chain. According to the present invention, a fatty chain has at least 8 carbon atoms, preferably from 8 to 30 carbon atoms, more preferably from 10 to 30 carbon atoms and especially from 10 to 22 carbon atoms.

According to the present invention, the cationic associative polymer has at least one repetitive unit other than an oxyalkylenated group. So associative polymers are different from products resulting merely from the condensation of an alkylene oxide with an alcohol, an ester or an amide.

The hydrophobically-modified cationic polymer or cationic amphiphilic associative polymers according to the present invention may be chosen from:
(1) cationic associative amphiphilic polyurethanes, which may be water-soluble or water-dispersible, the family of which has been described by the Applicant in French patent application No. 00/09609.

The cationic associative amphiphilic polyurethanes such as those may be represented by the general formula (AXIII) below:

R—X—(P)ₙ-[L-(Y)ₘ]ᵣ-L¹-(P′)ₚ—X′—R′ (AXIII)

in which:

R and R′, which may be identical or different, represent a hydrophobic group or a hydrogen atom;

X and X′, which may be identical or different, represent a group comprising an amine function optionally bearing a hydrophobic group, or alternatively a group L″;

L, L′ and L″, which may be identical or different, represent a group derived from a diisocyanate;

P and P′, which may be identical or different, represent a group comprising an amine function optionally bearing a hydrophobic group;

Y represents a hydrophilic group;

r is an integer between 1 and 100, preferably between 1 and 50 and in particular between 1 and 25;

n, m and p each range, independently of each other, between 0 and 1000; the molecule containing at least one protonated or quaternized amine function and at least one hydrophobic group.

In one preferred embodiment of these polyurethanes, the only hydrophobic groups are the groups R and R′ at the chain ends.

One preferred family of cationic amphiphilic associative polyurethanes is the one corresponding to formula (AXIII) described above and in which:

R and R′ both independently represent a hydrophobic group,

X and X′ each represent a group L″,

n and p are between 1 and 1000, and

L, L′, L″, P. P′, Y and m have the meaning given above.

Another preferred family of cationic associative polyurethanes is the one corresponding to formula (AXIII) above in which:

R and R′ both independently represent a hydrophobic group,

X and X′ each represent a group L″, n and p are 0, and

L, L′, L″, Y and m have the meaning given above.

The fact that n and p are 0 means that these polymers do not comprise units derived from a monomer containing an amine function, incorporated into the polymer during the polycondensation. The protonated amine functions of these polyurethanes result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide, a sulfate, etc.

Yet another preferred family of cationic associative polyurethanes is the one corresponding to formula (AXIII) above in which:

R and R′ both independently represent a hydrophobic group,

X and X′ both independently represent a group comprising a quaternary amine,

n and p are zero, and

L, L′, Y and m have the meaning given above.

The number-average molecular mass of the cationic associative polyurethanes is preferably between 400 and 500 000, in particular between 1000 and 400 000 and ideally between 1000 and 300000.

The expression “hydrophobic group” means a radical or polymer containing a saturated or unsaturated, linear or branched hydrocarbon-based chain, which may contain one or more heteroatoms such as P, O, N or S, or a radical containing a perfluoro or silicone chain. When the hydrophobic group denotes a hydrocarbon-based radical, it comprises at least 8 carbon atoms, preferably from 10 to 30 carbon atoms, in particular from 12 to 30 carbon atoms and more preferably from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote(s) a group comprising a tertiary or quaternary amine, X and/or X′ may represent one of the following formulae:

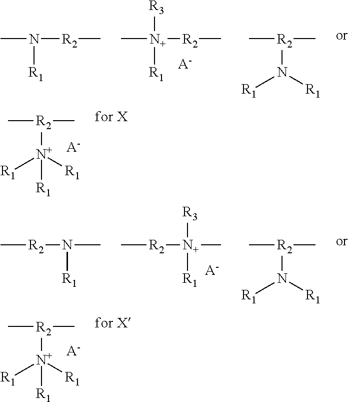

in which:

R₂ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P;

R₁ and R₃, which may be identical or different, denote a linear or branched C₁-C₃₀ alkyl or alkenyl radical or an aryl radical, at least one of the carbon atoms possibly being replaced with a heteroatom chosen from N, S, O and P; and

A- is a physiologically acceptable counter ion.

The groups L, L′ and L″ represent a group of formula:

Z—CO—NH—R₄—NH—CO—Z—

in which:

Z represents —O—, —S— or —NH—; and

R₄ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P.

The groups P and P′ comprising an amine function may represent at least one of the following formulae:

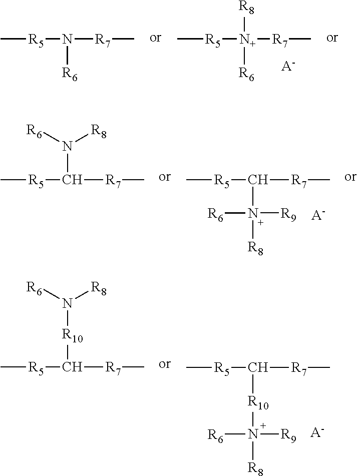

in which:

R₅ and R₇ have the same meanings as R₂ defined above;

R₆, R₈ and R₉ have the same meanings as R₁ and R₃ defined above;

R₁₀ represents a linear or branched, optionally unsaturated alkylene group possibly containing one or more heteroatoms chosen from N, O, S and P; and

A- is a physiologically acceptable counter ion.

As regards the meaning of Y, the term “hydrophilic group” means a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulfonated polyesters, sulfonated polyamides or a mixture of these polymers. The hydrophilic compound is preferentially a polyether and especially a poly(ethylene oxide) or poly(propylene oxide).

The cationic associative polyurethanes of formula (AXIII) that may be used according to the present invention are formed from diisocyanates and from various compounds bearing functions containing a labile hydrogen. The functions containing a labile hydrogen may be alcohol, primary or secondary amine or thiol functions, giving, after reaction with the diisocyanate functions, polyurethanes, polyureas and polythioureas, respectively. The expression “polyurethanes that may be used according to the present invention” encompasses these three types of polymer, namely polyurethanes per se, polyureas and polythioureas, and also copolymers thereof.

A first type of compound involved in the preparation of the polyurethane of formula (AXIII) is a compound comprising at least one unit containing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae:

HZ—(P)ₙ—ZH

or

HZ—(P′)ₚ—ZH

in which Z, P, P′, n and p are as defined above.

Examples of compounds containing an amine function that may be mentioned include N-methyldiethanolamine, N-tert butyldiethanolamine and N-sulfoethyldiethanolamine.

The second compound included in the preparation of the polyurethane of formula (AXIII) is a diisocyanate corresponding to the formula:

O═C═N—R₄—N═C═O

in which R₄ is as defined above. By way of example, mention may be made of methylenediphenyl diisocyanate, methylenecyclohexane diisocyanate, isophorone diisocyanate, tolylene diisocyanate, naphthalene diisocyanate, butane diisocyanate and hexane diisocyanate.

A third compound involved in the preparation of the polyurethane of formula (AXIII) is a hydrophobic compound intended to form the terminal hydrophobic groups of the polymer of formula (AXIII).

This compound is formed form a hydrophobic group and a function containing a labile hydrogen, for example a hydroxyl, primary or secondary amine, or thiol function. By way of example, this compound may be a fatty alcohol such as, in particular, stearyl alcohol, dodecyl alcohol or decyl alcohol.

When this compound comprises a polymeric chain, it may be, for example, α-hydroxylated hydrogenated polybutadiene.

The hydrophobic group of the polyurethane of formula (AI) may also result from the quaternization reaction of the tertiary amine of the compound comprising at least one tertiary amine unit. Thus, the hydrophobic group is introduced via the quaternizing agent.

This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine or thiol functions. This compound may be a polymer terminated at the chain ends with one of these functions containing a labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and especially a poly(ethyleneoxide) or poly(propylene oxide).

The hydrophilic group termed Y in formula (AXIII) is optional.

Specifically, the unit s containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred.
(2) quaternized cellulose derivatives.

The quaternized cellulose derivatives are, in particular quaternized celluloses, particularly quaternized hydroxyethyl celluloses modified with groups comprising at least one fatty chain, such as alkyl, arylalkyl or alkylaryl groups comprising at least 8 carbon atoms, or mixtures thereof.

The alkyl radicals borne by the above quaternized celluloses or hydroxyethyl celluloses preferably contain from 8 to 30 carbon atoms, especially from 10 to 30 carbon atoms. The aryl radicals preferably denote phenyl, benzyl, naphthyl or anthryl groups.

Examples of quaternized alkylhydroxyethylcelluloses containing C₈-C₃₀ fatty chains that may be mentioned include the products Quatrisoft LM 200, Quatrisoft LM-X 529-18-A, Quatrisoft LM-X 529-18B (C12 alkyl) and Quatrisoft LM-X 529-8 (C18 alkyl) or Softcat Polymer SL100, Softcat SX-1300X, Softcat SX-1300H, Softcat SL-5, Softcat SL-30, Softcat SL-60, Softcat SK-MH, Softcat SX-400X, Softcat SX-400H, SoftCat SK-L, Softcat SK-M, and Softcat SK-H, sold by the company Amerchol and the products Crodacel QM, Crodacel, QL (C12 alkyl) and Crodacel QS (C18 alkyl) sold by the company Croda.

Among these quaternized alkylhydroxyethylcelluloses the products corresponding to INCI name Polyquaternium 67 are preferred.

The quaternized cellulose derivatives could also be chosen among cationic cellulose ether(s), comprising from 4 000 to 10 000 anhydroglucose units, said anhydroglucose units being substituted with at least:

(i) one substituent of formula

[R₄R₅R₆R₉N⁺](X₂⁻)

in which

R₄ and R₅ represent, independently of one another, a methyl or ethyl group,

R₆ represents a linear or branched C₈-C₂₄ alkyl group or an aralkyl group in which the linear or branched alkyl part is C₈-C₂₄,

R₉ represents a divalent group which allows the attachment to the anhydroglucose group and which is chosen from —(B)_{q}—CH₂—CHOH—CH₂— and —CH₂CH₂—,

q denoting 0 or 1,

B denoting a divalent group —(CH₂CH₂O)ₙ—,

n′ being an integer ranging from 1 to 100,

X₂⁻ represents an anion; and

(ii) one substituent of formula

[R₁R₂R₃R₈N⁺](X₁⁻)

in which:

R¹, R² and R³ represent, independently of one another, a methyl or ethyl group,

R⁸ represents a divalent group which allows the attachment to the anhydroglucose group and which is chosen from -(A)ₚ-CH₂—CHOH—CH₂— and —CH₂CH₂—,

p denoting 0 or 1,

A denoting a divalent group —(CH₂CH₂O)ₙ—,

n being an integer ranging from 1 to 100,

X₁⁻ represents an anion.

Preferably, the substituent (i) of formula [R₄R₅R₆R₉N⁺](X₂) is present at an average of from 0.0003 to 0.08 mol, per mole of anhydroglucose units.

The cationic cellulose ethers that can be used in the compositions according to the present invention are preferably hydroxyethyl celluloses or hydroxypropyl celluloses. The cationic cellulose ethers that can be used in the compositions according to the present invention preferably comprise more than 4500, advantageously more than 5000, and more preferably more than 6000 anhydroglucose units.

Preferably, the cationic cellulose ethers that can be used in the compositions according to the present invention preferably comprise up to 9000, and preferably up to 8000 anhydroglucose units.

These cationic cellulose ethers and the process for the preparation thereof are described in application WO 2005/000903.

According to a preferred variant, the cationic cellulose ethers that can be used in the compositions according to the present invention are formed from at least one unit (IV) and at least one of the following units (I), (II) and (III):

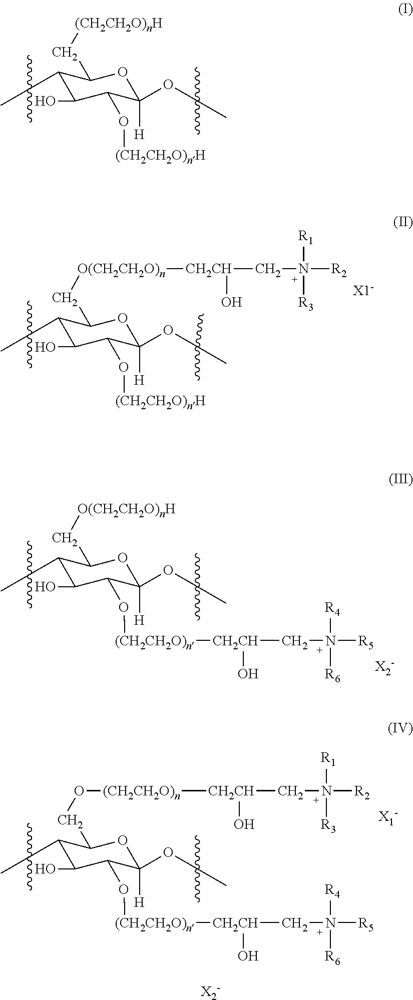

with the proviso that:

the total number of units (I)+(II)+(III)+(IV) is between 4000 and 10 000;

the [(III)+(IV)]/[(I)±(II)+(III)+(IV)] ratio ranges from 0.0003 to 0.8;

the [(II)±(IV)]/[(I)(II)+(III)±(IV)] ratio ranges from 0.02 to 0.9;

the integers n and n′, independently of one another, range from 0 to 5;

R₁, R₂, R₃, R₄ and R₅ represent, independently of one another, a methyl or ethyl group;

R₆ represents a linear or branched C₈-C₂₄, preferably C₁₀-C₂₄, more preferably C₁₂-C₂₄ and better still C₁₂-C₁₅, alkyl group or an aralkyl group in which the linear or branched alkyl part is C₈-C₂₄; X₁⁻ and X₂⁻ represent anions preferably chosen, independently of one another, from phosphate, nitrate, sulphate and halide (a, Br⁻, F⁻, I⁻) ions.

According to a particular variant, the cationic cellulose ethers that can be used in the compositions according to the present invention are formed from at least one unit (IV) and at least one of the units (I), (II) or (III) above, in which R₆ is a linear dodecyl group.

Among the cationic cellulose ethers that can be used in the compositions of the present invention, mention may be made of the polymers of Softcat SL-5, SL-30, SL-60 and SL-100 type (INCI: Polyquaternium-67) sold by the company Amerchol. The cationic cellulose ethers that are particularly preferred are the polymers of SL-60 and SL-100 type.
(3) cationic poly(vinyllactam) polymers which may comprise:
a) at least one monomer of vinyllactam or alkylvinyllactam type;
b) at least one monomer of structure (Ia) or (Ib) below:

CH₂═C(R₁)—CO—X—(Y)ₚ(CH₂—CH₂—O)ₘ—(CH₂—CH(R₂)—O)ₙ—(Y₁)_{q}—N⁺R₄R₃R₅Z⁻ (Ia)

CH₂═C(R₁)—CO—X—(Y)ₚ—(CH₂—CH₂—O)ₘ—(CH₂—CH(R₂)O)ₙ—(Y₁)_{q}—NR₃R₄ (Ib)

in which:

X denotes an oxygen atom or a radical NR₆,

R₁ and R₆ denote, independently of each other, a hydrogen atom or a linear or branched C₁-C₅ alkyl radical,

R₂ denotes a linear or branched C₁-C₄ alkyl radical,

R₃, R₄ and R₅ denote, independently of each other, a hydrogen atom, a linear or branched C₁-C₃₀ alkyl radical or a radical of formula (II):

—(Y₂)ᵣ—(CH₂—CH(R₇)—O)ₓ—R₈ (II)

Y, Y₁ and Y₂ denote, independently of each other, a linear or branched C₂-C₁₆ alkylene radical,

R₇ denotes a hydrogen atom or a linear or branched C₁-C₄ alkyl radical or a linear or branched C₁-C₄ hydroxyalkyl radical,

R₈ denotes a hydrogen atom or a linear or branched C₁-C₃₀ alkyl radical,

p, q and r denote, independently of each other, either the value 0 or the value 1,

m and n denote, independently of each other, an integer ranging from 0 to 100,

x denotes an integer ranging from 1 to 100,

Z denotes an organic or mineral acid anion, with the proviso that:
at least one of the substituents R₃, R₄, R₅ or R₈ denotes a linear or branched C₉-C₃₀ alkyl radical,
if m or n is other than zero, then q is equal to 1,
if m or n is equal to zero, then p or q is equal to 0.

The cationic poly(vinyllactam) polymers used in the composition according to the present invention may be crosslinked or noncrosslinked and may also be block polymers.

Preferably, the counter ion Z— of the monomers of formula (Ia) is chosen from halide ions, phosphate ions, the methosulfate ion and the tosylate ion.

Preferably, R₃, R₄ and R₅ denote, independently of each other, a hydrogen atom or a linear or branched C₁-C₃₀ alkyl radical.

More preferably, the monomer b) is a monomer of formula (Ia) for which, even more preferably, m and n are equal to 0.

The vinyllactam or alkylvinyllactam monomer is preferably a compound of structure (XV):

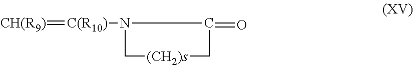

in which:

s denotes an integer ranging from 3 to 6,

R₉ denotes a hydrogen atom or a C₁-C₅ alkyl radical,

R₁₀ denotes a hydrogen atom or a C₁-C₅ alkyl radical,

with the proviso that at least one of the radicals R₉ and R₁₀ denotes a hydrogen atom. Even more preferably, the monomer (XV) is vinylpyrrolidone.

The cationic poly(vinyllactam) polymers used in the composition according to the present invention may also contain one or more additional monomers, preferably cationic or nonionic monomers. As compounds that are more particularly preferred according to the present invention, mention may be made of the following terpolymers comprising at least:

a) one monomer of formula (XV),

b) one monomer of formula (Ia) in which p=1, q=0, R₃ and R₄ denote, independently of each other, a hydrogen atom or a C₁-C₅ alkyl radical and R₅ denotes a C₉-C₂₄ alkyl radical, and

c) one monomer of formula (Ib) in which R₃ and R₄ denote, independently of each other, a hydrogen atom or a C₁-C₅ alkyl radical. Even more preferentially, terpolymers comprising, by weight, 40% to 95% of monomer (a), 0.1% to 55% of monomer (c) and 0.25% to 50% of monomer (b) will be used.

Such polymers are described in patent application WO 00/68282, the content of which forms an integral part of the present invent ion. As cationic poly(vinyllactam) polymers according to the present invention, vinylpyrrolidone/dimethylaminopropylmethacrylamide/dodecyldimethyl methacrylamido propylammonium tosylate terpolymers, vinyl pyrrolidone/dimethylamino propylmethacrylamide/cocoyldimethylmethacrylamidopropylammonium tosylate terpolymers, vinylpyrrolidone/dimethylaminopropyl methacrylamide/lauryldimethylmethacrylamido propylammoniumtosylate or chloride terpolymers are used in particular.

The weight-average molecular mass of the cationic poly(vinyllactam) polymers according to the present invention is preferably between 500 and 20 000 000. It is more particularly between 200 000 and 2 000 000, and even more preferably between 400 000 and 800 000.

One polymer that is particularly preferred is the polymer sold under the name Styleze W20 by the company ISP, which is a terpolymer of vinylpyrrolidone/dimethylaminopropylmethacrylamide and of lauryldimethylmethacrylamidopropylammonium chloride.
(4) cationic polymer(s) obtained by polymerization of a monomer mixture comprising one or more vinyl monomers substituted with one or more amino groups, one or more hydrophobic nonionic vinyl monomers, and one or more associative vinyl monomers.

In particular, among these cationic polymers, mention may be made especially of the compound sold by the company Noveon under the name Aqua CC and which corresponds to the INCI name Polyacrylate-1 Crosspolymer.

Polyacrylate-1 Crosspolymer is the product of polymerization of a monomer mixture comprising:
a di(C₁-C₄ alkyl)amino(C₁-C₆ alkyl) methacrylate,
one or more C₁-C₃₀ alkyl esters of (met h)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.

In particular, the cationic associative polymer is a cationic amphiphilic polymer having at least one fatty chain comprising at least 8 carbon atoms, especially from 10 to 30 carbon atoms and more specifically from 10 to 22 carbon atoms.

More preferably, the cat ionic associative polymer is chosen from:
quaternized celluloses, particularly quaternized hydroxyethylcelluloses modified with groups comprising at least one fatty chain, such as alkyl, arylalkyl or alkylaryl groups comprising at least 8 carbon atoms, or mixtures thereof, especially from 10 to 30 carbon atoms and more specifically from 10 to 22 carbon atoms.

In a more preferred embodiment, the cationic associative polymer is chosen from quaternized hydroxyethyl celluloses modified with at least an alkyl group containing at least 8 carbon atoms, especially from 10 to 22 carbon atoms and more specifically from 10 to 16 carbon atoms.

The cationic associative polymer is especially an associative cationic polymer having the INCI name POLYQUATERNIUM-67.

The (b) hydrophobically-modified cationic polymer is not used a surface modifier of the (c) water-insoluble particle described below. Thus, the (b) hydrophobically-modified cationic polymer is present in the cosmetic composition according to the present invention as an essential ingredient which is independent from the (a) anionic surfactant and the (c) water-insoluble particle.

According to the present invention, the amount of the (b) hydrophobically-modified cationic polymer may be 0.005% by weight or more, preferably 0.01% by weight or more, and more preferably 0.05% by weight or more, relative to the total weight of the composition. For example, the amount of the (b) hydrophobically-modified cationic polymer may range from 0.005% to 10% by weight, preferably from 0.01% to 5% by weight, and more preferably from 0.05% to 1% by weight, relative to the total weight of the composition.

[Water-Insoluble Particle]

The cosmetic composition according to the present invention comprises at least one water-insoluble particle, and two or more water-insoluble particles may be used in combination. Thus, a single type of water-insoluble particle or a combination of different types of water-insoluble particles may be used.

The water-insoluble particles according to the present invention comprise at least an inorganic part and/or a polymeric part and/or a visible light absorbing part.

The inorganic and/or polymeric and/or visible light absorbing part could be a part of the particle itself and/or could form part or all of a coating or surface treatment of the particle.

The (c) water-insoluble particle according to the present invention could then be chosen among:
water-insoluble particle comprising at least an inorganic part and/or a polymeric part and/or a visible light absorbing part, said particle being or not surface-treated;
water-insoluble particle comprising no inorganic part, no polymeric part and no visible light absorbing part, said particle being surface-treated with a compound comprising at least an inorganic part and/or at least a polymeric part and/or at least a visible light absorbing part.

For the purposes of the present invention, the term “water-insoluble particle” means a particle with a solubility in water at 25° C. of less than 1% by weight, preferably less than 0.1% by weight and more preferably less than 0.01% by weight, relative to the total weight of the particle, and most preferably with no solubility.

The diameter of the water-insoluble particles is not limited but may have a number-average particle size of 10 nm or more. The average particle size of the particles is preferably 50 nm or more, more preferably 100 nm or more, and even more preferably 200 nm or more, and is preferably 1000 μm or less, more preferably 100 μm or less, even more preferably 50 μm or less, and furthermore preferably 20 μm or less. Thus, it is possible that the water-insoluble particle has a particle size of from 10 nm to 1000 μm, preferably from 50 nm to 100 μm, more preferably from 0.1 to 50 μm, and more preferably from 0.2 to 20 μm. The number-average particle size may be measured by dynamic light scattering with, for example, Nicomp Z380.

The water-insoluble particles are preferably in the form of a solid. More preferably, the water-insoluble particles may be powders. The powders may be pigments and/or fillers.

The pigments preferably have an absorption ranging from 350 to 700 nm, and in at least one embodiment, an absorption with a maximum in this absorption range.

The pigments may be organic pigments. As used herein, the term “organic pigment” means any pigment that satisfies the definition in Ullmann's encyclopedia in the chapter on organic pigments. The organic pigment may be chosen, for example, from nitroso, nitro, azo, xanthene, quinoline, anthraquinone, phthalocyanin, metal complex, isoindolinone, isoindoline, quinacridone, perinone, perylene, diketopyrrolopyrrole, thioindigo, dioxazine, triphenylmethane, and quinophthalone compounds.

The at least one organic pigment may be chosen, for example, from carmine, carbon black, aniline black, melanin, azo yellow, quinacridone, phthalocyanin blue, sorghum red, the blue pigments codified in the Color Index under the references Cl 42090, 69800, 69825, 73000, 74100, and 74160, the yellow pigments codified in the Color Index under the references Cl 11680, 11710, 15985, 19140, 20040, 21100, 21108, 47000, and 47005, the green pigments codified in the Color Index under the references Cl 61565, 61570, and 74260, the orange pigments codified in the Color

Index under the references Cl 11725, 15510, 45370, and 71105, the red pigments codified in the Color Index under the references Cl 12085, 12120, 12370, 12420, 12490, 14700, 15525, 15580, 15620, 15630, 15800, 15850, 15865, 15880, 17200, 26100, 45380, 45410, 58000, 73360, 73915, and 75470, and the pigments obtained by oxidative polymerization of indole or phenolic derivatives as described, for example, in French Patent No. 2 679 771.

These pigments may also be in the form of composite pigments as described, for example, in European Patent No. 1 184 426. These composite pigments may be composed, for instance, of particles comprising an inorganic nucleus at least partially coated with an organic pigment and at least one binder to fix the organic pigments to the nucleus.

Other examples may include pigmentary pastes of organic pigments such as the products sold by the company Hoechst under the names: Jaune Cosmenyl IOG: Pigment Yellow 3 (Cl 11710); Jaune Cosmenyl G: Pigment Yellow 1 (Cl 11680); Orange Cosmenyl GR: Pigment Orange 43 (Cl 71105); Rouge Cosmenyl R″: Pigment Red 4 (Cl 12085); Carmine Cosmenyl FB: Pigment Red 5 (Cl 12490); Violet Cosmenyl RL: Pigment Violet 23 (Cl 51319); Bleu Cosmenyl A2R: Pigment Blue 15.1 (Cl 74160); Vert Cosmenyl GG: Pigment Green 7 (Cl 74260); and Noir Cosmenyl R: Pigment Black 7 (Cl 77266).

The at least one pigment may also be chosen from lakes. As used herein, the term “lake” means insolublized dyes adsorbed onto insoluble particles, the complex or the compound thus obtained remaining insoluble during use.

The inorganic substrates onto which the dyes are adsorbed may include, for example, alumina, silica, calcium sodium borosilicate, calcium aluminum borosilicate, and aluminum.

Non-limiting examples of organic dyes include cochineal carmine and the products known under the following names: D&C Red 21 (Cl 45 380), D&C Orange 5 (Cl 45 370), D&C Red 27 (Cl 45 410), D&C Orange 10 (Cl 45 425), D&C Red 3 (Cl 45 430), D&C Red 4 (Cl 15 510), D&C Red 33 (Cl 17 200), D&C Yellow 5 (Cl 19 140), D&C Yellow 6 (Cl 15 985), D&C Green (Cl 61 570), D&C Yellow 10 (Cl 77 002), D&C Green 3 (Cl 42 053), and D&C Blue 1 (Cl 42 090).

An additional non-limiting example of a lake is the product known under the following name: D&C Red 7 (Cl 15 850:1).

The at least one pigment may also be a pigment with special effects. As used herein, the term “pigments with special effects” means pigments that generally create a non-uniform colored appearance (characterized by a certain shade, a certain vivacity, and/or a certain lightness) that changes as a function of the conditions of observation (light, temperature, observation angles, etc.). They thus contrast with white or colored pigments that afford a standard uniform opaque, semi-transparent, or transparent shade.

Two types of pigment with special effects exist: those with a low refractive index, such as fluorescent, photochromic, and thermochromic pigments, and those with a high refractive index, such as nacres and flakes.

The at least one pigment may also be chosen from pigments with an interference effect that are not fixed onto a substrate, for instance, liquid crystals (Helicones HC from Wacker), and holographic interference flakes (Geometric Pigments or Spectra f/x from Spectratek). Pigments with special effects may also comprise fluorescent pigments, whether these are substances that are fluorescent in daylight or that produce an ultraviolet fluorescence, phosphorescent pigments, photochromic pigments, thermochromic pigments, and quantum dots, sold, for example, by the company Quantum Dots Corporation.

The pigments with special effects may also comprise fluorescent pigments, whether these are substances that are fluorescent in daylight or that produce an ultraviolet fluorescence, phosphorescent pigments, photochromic pigments, and thermochromic pigments.

The pigment may also be an inorganic pigment, in a preferred embodiment. As used herein, the term “inorganic pigment” means any pigment that satisfies the definition in Ullmann's encyclopedia in the chapter on inorganic pigments. Preferably, the inorganic pigments comprise at least one inorganic material. Non-limiting examples of inorganic pigments that are useful in the present invention include metal oxides, in particular, transition metal oxides, such as zirconium oxides, cerium oxides, iron oxides, chromium oxides, manganese violet, ultramarine blue, chromium hydrate, ferric blue, and titanium dioxide. The following inorganic pigments may also be used: Ta₂O₅, Ti₃O₅, Ti₂O₃, TiO, and ZrO₂ as a mixture with TiO₂, ZrO₂, Nb₂O₅, CeO₂, and ZnS.

The pigment may also be a nacreous pigment such as a white nacreous pigment, for example, mica coated with titanium or with bismuth oxychloride, a colored nacreous pigment such as mica coated with titanium and with iron oxides, mica coated with titanium and, for example, with ferric blue or chromium oxide, mica coated with titanium and with an organic pigment as defined above, and also a nacreous pigment based on bismuth oxychloride. Examples of such pigments may include the Cellini pigments sold by Engelhard (Mica-TiO₂-lake), Prestige sold by Eckart (Mica-TiO₂), and Colorona sold by Merck (Mica-TiO₂—Fe₂O₃).

In addition to nacres on a mica support, multilayer pigments based on synthetic substrates such as alumina, silica, calcium sodium borosilicate, calcium aluminum borosilicates, and aluminum, may be useful in accordance with the present disclosure.

As used herein, the term “filler” means a substantially uncolored compound that is solid at room temperature and atmospheric pressure, and insoluble in the various ingredients of the cosmetic composition according to the present invention, even when these ingredients are brought to a temperature above room temperature.

The filler may be chosen from mineral and organic fillers. When the fillers are organic fillers, they are polymeric organic fillers. The filler may be particles of any form, for example, platelet-shaped, spherical, and oblong, irrespective of their crystallographic form (for example lamellar, cubic, hexagonal, and orthorhombic).

The fillers that may be used in the cosmetic composition according to the present invention can be made from various inorganic and/or organic materials, and may include, but are not limited to, titanium dioxide; talc; natural or synthetic mica; alumina; aluminosilicate; silica (or silicon dioxides); kaolin or other insoluble silicates such as clays; polyamides (Nylon®), poly-β-alanine and polyethylene powders; tetrafluoroethylene polymer (Teflon®) powders starch; boron nitride; acrylic acid polymer powders; silicone resin microbeads, for instance “Tospearls®” from the company Toshiba; bismuth oxychlorides; precipitated calcium carbonate; magnesium carbonate and magnesium hydrogen carbonate; hydroxyapatite; hollow silica microspheres such as “Silica Beads SB 700®” and “Silica Beads SB 7000” from the company Maprecos, “Sunspheres H-33®” and “Sunspheres H-51Z” from the company Asahi Glass; acrylic polymer microspheres such as those made from crosslinked acrylate copolymer “Polytrap 6603C)” from the company R.P. Scherrer and those made from polymethyl methacrylate “Micropearl M100®” from the company SEPPIC; polyurea powders; polyurethane powders such as the hexamethylene diisocyanate and trimethylol hexyl lactone copolymer powder sold under the name “Plastic Powder D-4000” by the company Toshiki; glass or ceramic microcapsules; microcapsules of methyl acrylate or methacrylate polymers or copolymers, or alternatively, vinylidene chloride and acrylonitrile copolymers, for instance, “Expancel®” from the company Expancel; elastomeric crosslinked organopolysiloxane powders such as those sold under the name “KSP100®” by the company Shinetsu Chemical; porous cellulose beads such as those sold under the name of Cellulose Beads USF® by the company Daito Kasei; and mixtures thereof.

It is preferable that the (c) water-insoluble particle may be in the form of a microcapsule or a microsphere. The microcapsule is in the form of a very small hollow capsule which can include a substance therein. The microsphere is in the form of a very small sphere which can include a substance, preferably a liquid substance, while it is, in general, not hollow but solid, due to very small pores formed in the sphere (porous material). The microsphere may have a hollow therein.

Among the silicas that are useful in the composition of the present invention, mention may be made of crystalline, microcrystalline and non-crystalline silicas.

By way of example, crystalline silicas that may be mentioned include quartz, tridymite, cristobalite, keatite, coesite and stishovite. The microcrystalline silicas are, for example, diatomite.

Among the non-crystalline forms that may be used are vitreous silica and other types of amorphous silicas such as colloidal silicas, silica gels, precipitated silicas and fumed silicas, for instance aerosils, and pyrogenic silicas. Porous silica such as an aerogel (silica silylate) is preferable.

In one embodiment of the present invention, the (c) water-insoluble particle may comprise at least one inorganic material selected from the group consisting of talc, mica, silica, kaolin, sericite, calcinated talc, calcinated mica, calcinated sericite, synthetic mica, bismuth oxychloride, barium sulfate, boron nitride, calcium carbonate, magnesium carbonate, magnesium hydrogen carbonate, and hydroxyapatite. The (c) water-insoluble particle may comprise selenium disulfide.

In another embodiment of the present invention, the (c) water-insoluble particle may comprise at least one organic material selected from the group consisting of polyurea, melamine-formaldehyde condensate, urea-formaldehyde condensate, aminoplasts, polyurethane, polyacrylate, polyphosphate, polystyrene, polyester, polyamide, polyolefin, polysaccharide, silicone, silicone resin, protein, modified cellulose, and gum.

In another embodiment particles may be organomineral particles such as zinc pyrithione particles.

Particularly suitable water-insoluble particles for use in the present invention are microcapsules or microspheres (oil delivery agent or oil absorber). The preferred material of the particles is at least one of polyurea, melamine-formaldehyde condensate, urea-formaldehyde condensate, aminoplasts, polyurethane, polyacrylate, polyphosphate, polystyrene, polyester, polyamide, polyolefin, polysaccaharide, silica, silicone resin, protein, modified cellulose, gum, mica, talc, kaolin, carbonate and the like.

According to the present invention, the above-described water-insoluble particles may be surface treated. The surface treatment can be performed by any conventional process.

For the purposes of the present invention, the surface treatment is such that a surface-treated pigment conserves its intrinsic pretreatment pigmenting properties and a surface-treated filler conserves its intrinsic pretreatment filling properties. For example, the inorganic substrates such as alumina and silica onto which organic dyes are adsorbed are preferably not surface-treated fillers for the purposes of the present invention.

The water-insoluble particles may have at least one hydrophobic coating.

The hydrophobic coating may be formed by treating the water-insoluble particles with a hydrophobic treating agent. The hydrophobic treating agent can be chosen from silicones, such as methicones, dimethicones or perfluoroalkylsilanes; fatty acids, such as stearic acid; perfluoroalkyl phosphates, perfluoroalkylsilanes, perfluoroalkylsilazanes, poly(hexafluoropropylene oxides), polyorganosiloxanes comprising perfluoroalkyl or perfluoropolyether groups, and amino acids; N-acylated amino acids or their salts; lecithin, isopropyl triisostearyl titanate, and their mixtures.

As the water-insoluble particles, silicone-treated silica beads can be used.

As the water-insoluble particles, TiO₂ particles coated with at least one hydrophobic coating can also be used. Among the coated TiO₂ particles, mention may be made of:
those coated with polydimethylsiloxane (CARDRE ULTRAFINE TITANIUM DIOXIDE AS provided by the company CARDRE);
those coated with polymethylhydrogenosiloxane (untreated titanium oxide coated with polymethylhydrogenosiloxane sold under the trade name Cosmetic White SA-C47-051-10 by the company MYOSHI);
those coated with perfluoropolymethyl isopropyl ether (CARDRE MICA FHC 70173 OR 70170 CARDRE UF TIO2 FHC provided by the company CARDRE);
those coated with silica (SPHERITITAN AB provided by the company CATALYSTS & CHEMICALS;
those coated with teflon (CS-13997 TEFLON COATED TITANIUM DIOXIDE provided by the company CLARK COLORS); and
those coated with polyester (EXPERIMENTAL DESOTO BEADS provided by the company DESOTO).

Among these TiO₂ treated particles, TiO₂ particles coated with silicone such as polydimethylsiloxane are more preferable.

According to one embodiment of the present invention, the water-insoluble particles may be surface treated with at least one amphiphilic agent, in particular, the above-described water-insoluble particles may be partially or fully surface treated with at least one amphiphilic agent. It is preferable that the particles are partially treated with the amphiphilic agent(s). The water-insoluble particles may be located between the continuous phase and the dispersed phase of the cosmetic composition according to the present invention, to form a Pickering emulsion. The dispersed phases preferably connect with each other via the particles.

The amphiphilic agent can provide particles with both hydrophilic and hydrophobic properties. Preferably, the particles have an amphiphilic surface.

The amphiphilic agent may comprise at least one compound chosen from, for example, amino acids; fatty acids, fatty alcohols and derivatives thereof, such as stearic acid, hydroxystearic acid, stearyl alcohol, hydroxystearyl alcohol, lauric acid, and derivatives thereof; anionic surfactants; lecithins; sodium, potassium, magnesium, iron, titanium, zinc, or aluminum salts of fatty acids, for example, aluminum stearate or laurate; metal alkoxides; polysaccharides, for example, chitosan, cellulose, and derivatives thereof; polyethylenes; (meth)acrylic polymers, for example, polymethyl methacrylates; polymers and copolymers containing acrylate units; proteins; and alkanolamines.

The particles may be surface treated with a mixture of amphiphilic agents, and/or may be subjected to several surface treatments with amphiphilic agents.

The surface-treated particles may be prepared according to surface-treatment techniques that are well known to those skilled in the art, or may be commercially available in the required form.

Preferably, the surface-treated particles are coated with an organic layer. The organic layer may be deposited on the particles by evaporation of a solvent, chemical reaction between the molecules in the amphiphilic agents, or creation of a covalent bond between the molecules in the amphiphilic agents and the particles.

The surface treatment may thus be performed, for example, by chemical reaction of the amphiphilic agent with the surface of the particles and creation of a covalent bond between the amphiphilic agent and the particles. This method is specifically described in U.S. Pat. No. 4,578,266.

Particles to which the amphiphilic agents covalently or ionically bond are preferably used.

The amphiphilic agents may represent from 0.1% to 50% by weight, preferably from 0.5% to 30% by weight, and more preferably 1% to 10% by weight, relative to the total weight of the surface-treated particles.

It is preferable that the amphiphilic agent comprises at least one hydrophobicized amino acid. The hydrophobicized amino acid may be a glutamic acid derivative or a condensate of at least one glutamic acid derivative and an amino acid.

The glutamic acid derivative may be N-acylated glutamic acid or a salt thereof. As the salt, mention may be made of metal salts, ammonium salts, and onium salts of an organic alkanolamine. As the metal, Na, K, Ba, Zn, Ca, Mg, Fe, Zr, Co, Al, and Ti may be used. As the organic alkanolamine, monoethanolamine, diethanolamine, triethanolamine, 2-amino-2-methylpropanol, 2-amino-2-methyl-1,3-propanediol, and triisopropanolamine may be used. The acyl group bound to the nitrogen atom of the glutamic acid may be derived from a saturated or unsaturated fatty acid having 8 to 22 carbon atoms, such as capric acid, lauric acid, myristic acid, isomyristic acid, palmitic acid, isopalmitic acid, stearic acid, isostearic acid, arachic acid, undecylenic acid, oleic acid, myristic acid, elaidic acid, linolic acid, linoleic acid, arachidonic acid, palm oil fatty acid, beef tallow fatty acid, and resin acid (abietic acid).

The condensate of at least one glutamic acid derivative and an amino acid may be a condensate of N-acylated glutamic acid and an amino acid such as lysine, or a salt thereof. As the salt, mention may be made of metal salts, ammonium salts and onium salts of an organic alkanolamine as mentioned above. Sodium salt is preferable. The acyl group bound to the nitrogen atom of the glutamic acid may be derived from a saturated or unsaturated fatty acid having 8 to 22 carbon atoms as mentioned above. Lauric acid is preferable. Thus, for example, sodium dilauramidoglutamide lysine (Pellicer L-30 marketed by Asahi Kasei Chemicals) is preferable as the above condensate.

The amphiphilic surface treatments of the particles may be chosen from the following treatments:
a PEG-silicone treatment, for instance, the AQ surface treatment sold by LCW;
a lauroyllysine treatment, for instance, the LL surface treatment sold by LCW;
a lauroyllysine dimethicone treatment, for instance, the LL/SI surface treatment sold by LCW;
a disodium stearoyl glutamate treatment, for instance, the NM surface treatment sold by Miyoshi;
a dimethicone/disodium stearoyl glutamate treatment, for instance, the SA/NAI surface treatment sold by Miyoshi;
a microcrystalline cellulose and carboxymethylcellulose treatment, for instance, the AC surface treatment sold by Daito;
an acrylate copolymer treatment, for instance, the APD surface treatment sold by Daito;
a sodium dilauramidoglutamide lysine treatment, for instance, the ASL treatment sold by Daito; and
a sodium dilauramidoglutamide lysine/isopropyl titanium triisostearate treatment, for instance, the ASL treatment sold by Daito.

Amphiphilic agent(s) can be bound to particles ionically with a metal salt or hydroxide whose metal can be selected from Mg, Al, Ca, and Zn, for instance, aluminum hydroxide and magnesium chloride.

A treatment using disodium stearoyl glutamate (and) aluminum hydroxide is more preferable.

Other treatments using a sodium dilauramidoglutamide lysine, or a sodium dilauramidoglutamide lysine/isopropyl titanium triisostearate, are also more preferable.

In one embodiment of the present invention, the (c) water-insoluble particle itself may function as a cosmetic active agent such as opacifiers, pearlescent agents, feel modifiers, skin protectants, matting agents, friction enhancers, slip agents, conditioning agents, exfoliants, odor absorbers, colouring agents and cleaning enhancers.

In another embodiment of the present invention, the (c) water-insoluble particle may include at least one additional cosmetic active agent particularly when they are microcapsules or microspheres.

There is no limit to the additional cosmetic active agent. The cosmetic active agent may be in the form of a solid or liquid at 25° C. under 1 atm. Two or more cosmetic active agents may be used in combination. Thus, a single type of cosmetic active agent or a combination of different types of cosmetic active agents may be used.

The cosmetic active agent may be selected from, for example, fragrance, conditioning agents, sunscreens (UV filters), anti-perspirant agents, anti-dandruff agents, and anti-bacterial agents. It is preferable that the cosmetic active agent does not include (poly)glycol (di)stearate.

In one embodiment, preferably the additional cosmetic ingredient is a fragrance.

As examples of the fragrance, a natural or synthetic fragrance or aroma, or a mixture thereof can be employed.

As examples of natural fragrances and aromas, mention may be made of, for example, extracts of flowers (lily, lavender, rose, jasmine, or ylang-ylang), extracts of stems and of leaves (patchouli, geranium, or petit grain), extracts of fruits (coriander, anise, caroway, or juniper), extracts of fruit rinds (bergamot, lemon, or orange), extracts of roots (angelica, celery, cardamom, iris, or sweet flag), extracts of wood (pinewood, sandalwood, lignum vitae, or pink cedar), extracts of gasses and of gramineous plants (tarragon, lemon grass, sage, or thyme), extracts of needle leaves and of branches (spruce, fir, pine, or dwarf pine), extracts of resins and of balms (galbanum, elemi, benzoin, myrrh, olibanum, or opopanax), and the like.

As examples of synthetic fragrances and aromas, mention may be made of, for example, esters, ethers, aldehydes, ketones, aromatic alcohols, and hydrocarbon-based compounds.

As specified examples of the aforementioned esters, mention may be made of benzyl acetate, benzyl benzoate, phenoxyethyl isobutyrate, p-t-butylcyclohexyl acetate, citronellyl acetate, citronellyl formate, geranyl acetate, linalyl acetate, dimethylbenzylcarbonyl acetate, phenylethyl acetate, linalyl benzoate, benzyl formate, ethylmethylphenyl glycinate, alkylcyclohexyl propionate, styralyl propionate, benzyl salicylate, and the like.

As examples of the aforementioned ethers, mention may be made of benzyl ethyl ether and the like.

As examples of the aforementioned aldehydes, mention may be made of, for example, linear alkanals having 8 to 18 carbon atoms, citral, citronellal, citronellyl oxyacetaldehyde, cyclamen aldehyde, hydroxycitronellal, lilial, bourgeonal, and the like.

As examples of the aforementioned ketones, mention may be made of, for example, ionones such as α-isomethylionone and methyl cedryl ketone.

As examples of the aforementioned aromatic alcohols and in particular, terpene alcohols, mention may be made of anethole, citronellol, eugenol, isoeugenol, geraniol, linalol, phenylethyl alcohol, terpineol, and the like.

As examples of the aforementioned hydrocarbon-based compounds, mention may be made of, in particular, terpenes. The aforementioned compounds are often provided in the form of a blended product having two or more odorous substances in many cases.

An essential oil can also be employed as an aroma component. For example, sage oil, chamomile oil, clove oil, balm oil, mint oil, cinnamon leave oil, lime blossom oil, juniper oil, vetiver oil, olibanum oil, galbanum oil, labolanum oil, lavandin oil, and the like are employed.

In addition, the perfumes described below can be employed alone or in combination. Bergamot oil, dihydromyrcenol, lilial, lyral, citronellol, phenylethyl alcohol, α-hexylcinnamaldehyde, geraniol, benzylacetone, cyclamen aldehyde, linalol, ambroxan, indole, hedione, sandelice, lemon oil, oils from mandarins and oranges, allyl amine glycolate, cyclovertal, lavender oil, sage oil, β-damascone, geranium oil, cyclohexyl salicylate, phenylacetic acid, geranyl acetate, benzyl acetate, rose oxide, or the like, can be employed.

In accordance with a preferable mode for carrying out the present invention, various fragrances can be employed by blending the same. Thereby, a scent which is pleasing to the user can be obtained.

As examples of the conditioning agents, mention may be made of branched or unbranched, volatile or non-volatile linear or cyclic silicones. These silicones may be in the form of oils, resins or gums, they may in particular be polyorganosiloxanes that are insoluble in the cosmetically acceptable medium. Organopolysiloxanes are defined in greater detail in the work by Walter Noll, “Chemistry and Technology of Silicones” (1968) Academic Press. They may be volatile or non-volatile. When they are volatile, the silicones are more particularly chosen from those having a boiling point between 60° C. and 260° C. By way of conditioning agent, use can also be made of polymers such as the polyquaterniums 22, 6, 10, 11, 35 and 37 and hexadimethrine chloride.

As examples of the sunscreens, mention may be made of inorganic UV filter which may be selected from the group consisting of silicon carbide, metal oxides and mixtures thereof, as well as organic UV filters which may be selected from the group consisting of anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from α-alkylstyrene; 4,4-diarylbutadienes compounds; and mixtures thereof.

As examples of the anti-perspirant agent, mention may be made of aluminum salts, zirconium salts and zinc salts as mentioned above. Antiperspirant aluminum salts are preferable. As used herein, the term “antiperspirant aluminum salt” means any salt or any aluminum complex that has the effect of reducing or limiting the flow of sweat. The aluminum salt in accordance with the present disclosure may be, for example, selected from aluminum halohydrates; aluminum zirconium halohydrates; and complexes of zirconium hydroxychloride and of aluminum hydroxychloride with an amino acid, such as those described in U.S. Pat. No. 3,792,068, which are commonly known as “ZAG complexes”. Among the aluminum salts that may be mentioned, for example, are aluminum chlorohydrate in activated or unactivated form, aluminum chlorohydrex, aluminum chlorohydrex polyethylene glycol complex, aluminum chlorohydrex propylene glycol complex, aluminum dichlorohydrate, aluminum dichlorohydrex polyethylene glycol complex, aluminum dichlorohydrex propylene glycol complex, aluminum sesquichloro-hydrate, aluminum sesquichlorohydrex polyethylene glycol complex, aluminum sesquichlorohydrex propylene glycol complex, and aluminum sulfate buffered with sodium aluminum lactate. Among the aluminum zirconium double salts that may be mentioned, for example, are aluminum zirconium octachlorohydrate, aluminum zirconium pentachloro-hydrate, aluminum zirconium tetrachlorohydrate, and aluminum zirconium trichlorohydrate. An example of an aluminum zirconium double salt is the product sold by the company Reheis under the name Reach AZP-908-SUF. The complexes of zirconium hydroxychloride and of aluminum hydroxychloride with an amino acid are generally known under the name ZAG (when the amino acid is glycine). Among these products, mention may be made of the aluminum zirconium octachlorohydrex glycine, aluminum zirconium pentachlorohydrex glycine, aluminum zirconium tetrachlorohydrex glycine, and aluminum zirconium trichlorohydrex glycine complexes.

As examples of the anti-dandruff agents, mention may be made of the following.

1) Pyridinethione salts, especially the calcium, magnesium, barium, strontium, zinc, cadmium, tin and zirconium salts. The zinc salt of pyridinethione is particularly preferred. The zinc salt of pyridinethione is sold especially under the name Omadine zinc by the company Arch Personal Care.

2) 1-Hydroxy-2-pyrrolidone derivatives represented especially by formula (XXIX):

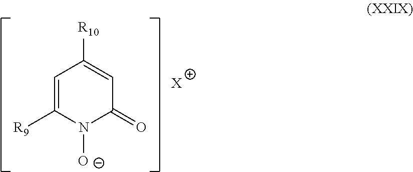

in which

R₉ represents an alkyl group containing from 1 to 17 carbon atoms, an alkenyl group containing from 2 to 17 carbon atoms, a cycloalkyl group containing from 5 to 8 carbon atoms, a bicycloalkyl group containing from 7 to 9 carbon atoms; a cycloalkyl (-alkyl) group, an aryl group, an aralkyl group with an alkyl containing from 1 to 4 carbon atoms, an arylallcenyl group with an alkenyl containing from 2 to 4 carbon atoms, aryloxyalkyl or arylmercaptoalkyl with an alkyl containing from 1 to 4 carbon atoms, a furylalkenyl group with an alkenyl or a furyl containing from 2 to 4 carbon atoms, an alkoxy group containing from 1 to 4 carbon atoms, a nitro group, a cyano group or a halogen atom;

R₁₀ represents a hydrogen atom, a C₁-C₄ alkyl group, a C₂-C₄ alkenyl group, a halogen atom, a phenyl group, or a benzyl group; Y represents an organic base, an alkali metal or alkaline-earth metal ion or an ammonium ion.

Examples of the compounds of formula (XXIX) include 1-hydroxy-4-methyl-2-pyridone, 1-hydroxy-6-methyl-2-pyridone, 1-hydroxy-4,6-dimethyl-2-pyridone, 1-hydroxy-4-methyl-6-(2,4,4-trimethylpentyl)-2-pyridone, 1-hydroxy-4-methyl-6-cyclohexyl-2-pyridone, 1-hydroxy-4-methyl-6-(methylcyclohexyl)-2-pyridone, 1-hydroxy-4-methyl-6-(2-bicyclo[2,2,1]heptyl)-2-pyridone, 1-hydroxy-4-methyl-6-(4-methylphenyl)-2-pyridone, 1-hydroxy-4-methyl-6-[1-(4-nitrophenoxy)butyl]-2-pyridone, 1-hydroxy-4-methyl-6-(4-cyanophenoxymethyl)-2-pyridone, 1-hydroxy-4-methyl-6-(phenylsulfonylmethyl)-2-pyridone and 1-hydroxy-4-methyl-6-(4-bromobenzyl)-2-pyridone.

The compounds of formula (XXIX) may be used in the form of salts with organic or mineral bases.

Examples of organic bases are especially alkanolamines of low molecular weight such as ethanolamine, diethanolamine, N-ethylethanolamine, triethanolamine, diethylaminoethanol and 2-amino-2-methylpropanediol; non-volatile bases such as ethylenediamine, hexamethylenediamine, cyclohexylamine, benzylamine and N-methylpiperazine; quaternary ammonium hydroxides, e.g. trimethylbenzyl hydroxide; guanidine and derivatives thereof, and particularly alkyl derivatives thereof. Examples of mineral bases are especially salts of alkali metals, e.g. sodium or potassium; ammonium salts, salts of an alkaline-earth metal, e.g. magnesium or calcium; salts of di-, tri- or tetravalent cationic metals, e.g. zinc, aluminium or zirconium. Alkanolamines, ethylenediamine and mineral bases such as alkali metal salts are preferred.

A compound of formula (XXX) that is particularly preferred is the one for which R₉ denotes the radical

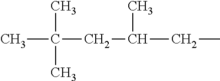

R₁₀ denotes methyl, and

X⁺ denotes N⁺H₃CH₂CH₂OH. This compound is sold, for example, under the name Octopirox (1-hydroxy-4-methyl-6-(2,4,4-trimethylpentyl)-2-pyridone, monoethanolamine salt) by the company Hoechst.

3) 2,2′-Dithiobis(pyridine N-oxide) of formula (XXX):

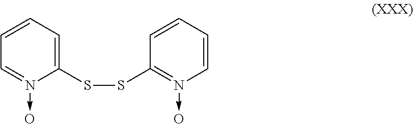 [SMILES: [O-][n+]1ccccc1SSc1cccc[n+]1[O-]]

The compounds of formula (XXX) may be introduced into the compositions in the form of mineral salts. An example of a mineral salt is magnesium sulfate.

4) Trihalocarbamides especially of formula (XXXI) below:

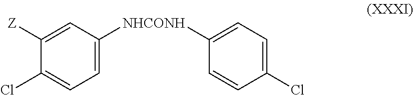

in which Z represents a halogen atom such as chlorine or a C1-C4 trihaloalkyl group such as CF3.

5) Triclosan, represented by formula (XXXII):

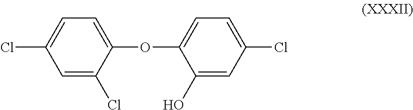 [SMILES: Oc1cc(Cl)ccc1Oc1ccc(Cl)cc1Cl]

6) Azole compounds such as climbazole, ketoconazole, clotrimazole, econazole, isoconazole and miconazole.

7) Selenium sulfides, in particular those of formula SₓSe₈₋ₓ, x ranging from 1 to 7.

8) Extracts of one or more non-photosynthetic, non-fruiting filamentous bacteria.

The bacterial extracts that may be used according to the present invention will be chosen from non-photosynthetic, non-fruiting filamentous bacteria as defined according to the classification in Bergey's Manual of Systemic Bacteriology, volume 3, section 23, 9th edition 1989.

Among the bacteria that may be used, mention will be made more particularly of bacteria belonging to the order Beggiatoales, and especially bacteria belonging to the genus Beggiotoa, for instance various strains of Beggiotoa alba. According to the definition, B. alba corresponds to the former names Beggiotoa arachnoidea, B. gigantea, B. leptomiformis, B. minima and B. mirabilis of Bergey's manual, 8th edition. Mention may moreover be made of bacteria belonging to the genus Vitreoscilla, which is known to be close to and often difficult to distinguish from the genus Beggiatoa. The bacteria that have just been defined, and several of which have been described, generally have an aquatic habitat, and may be found especially in spring water sources.

Among the bacteria that may be used, mention may be made, for example, of Vitreoscilla beggiatoides (ATCC 43181) and Beggiatoa alba (ATCC33555). According to the present invention, the use of the extract of Vitreoscilla filiformis, in particular the strain ATCC 15551, metabolites thereof and fractions thereof, may be preferred.

Moreover, it is known that culturing non-photosynthetic, non-fruiting filamentous bacteria is relatively difficult, as is the production of pure cultures. Use will preferentially be made of the culture described in patent application WO 94/02158.

The term “non-photosynthetic, non-fruiting filamentous bacteria” means not only the culture supernatant but also the biomass obtained after culturing the said bacteria, the envelopes or envelope fractions, or the extracts of the biomass obtained by treating this biomass.

To prepare the extract according to the present invention, the said bacteria can be cultured and then separated from the biomass obtained, for example by filtration, centrifugation, coagulation and/or lyophilization.

The extracts that may be used may especially be prepared according to the process described in patent application WO-A-93/00741. Thus, after culturing, the bacteria are concentrated by centrifugation. The biomass obtained is autoclaved. This biomass may be lyophilized to constitute what is known as the lyophilized extract. Any lyophilization method known to those skilled in the art may be used to prepare this extract.

The supernatant fraction of this biomass may also be filtered in a sterile container to remove the particles in suspension.

The terms “envelopes” and “envelope fractions” refer herein to the bacterial wall and possibly the subjacent membranes.

9) Antifungal polymers such as amphotericin B or nystatin.

10) Other antidandruff agents are sulfur in its various forms, cadmium sulfide, allantoin, coal or wood tars and derivatives thereof, in particular cade oil, salicylic acid, undecylenic acid, fumaric acid, ellagic acid, ellagic acid tannins, and allylamines such as terbinafine.

As examples of the anti-bacterial agent, mention may be made of 2,4,4′-trichloro-2′-hydroxydiphenyl ether (or triclosan), 3,4,4′-trichlorocarbanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopiroxe, ciclopiroxol-amine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, octopirox, octoxyglycerol, octanolglycine, caprylyl glycol, 10-hydroxy-2-decanoic acid, dichlorophenyl imidazole dioxolane and its derivatives described in Patent WO9318743, copper pidolate, salicylic acid, iodopropynyl butylcarbamate, farnesol, phytosphingosines and mixtures thereof. In addition, metal salts which can provide metal ions such as silver ions may be used. The scope of the anti-bacterial agent and the scope of the anti-dandruff agent may partially overlap.

It is preferable that the (c) water-insoluble particle include at least one cosmetic active agent, preferably a fragrance, a conditioning agent or a UV filter, and more preferably a fragrance.

The amount of the (c) water-insoluble particles may range from 0.01 to 10% by weight, preferably from 0.05 to 5% by weight, and more preferably from 0.1 to 3% by weight, relative to the total weight of the composition.

[Cosmetic Composition]

The cosmetic composition according to the present invention may comprise at least one optional ingredient not included in particles. These optional ingredients may be the same as additional ingredients for particles mentioned above. They may be also chosen from cationic and amphoteric surfactants.

(Cationic Surfactant)

The cosmetic composition according to the present invention may comprise at least one cationic surfactant, and two or more cationic surfactants may be used in combination. Thus, a single type of cationic surfactant or a combination of different types of cationic surfactants may be used.

The cationic surfactant is not limited. The cationic surfactant may be selected from the group consisting of optionally polyoxyalkylenated, primary, secondary or tertiary fatty amine salts, quaternary ammonium salts, and mixtures thereof.

Examples of quaternary ammonium salts that may be mentioned include, but are not limited to: those of general formula (I) below:

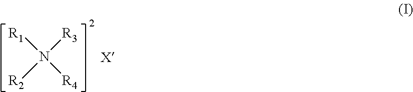

wherein

R₁, R₂, R₃, and R₁, which may be identical or different, are chosen from linear and branched aliphatic radicals comprising from 1 to 30 carbon atoms and optionally comprising heteroatoms such as oxygen, nitrogen, sulfur and halogens. The aliphatic radicals may be chosen, for example, from alkyl, alkoxy, C₂-C₆ polyoxyalkylene, alkylamide,

(C₁₂-C₂₂)alkylamido(C₂-C₆)alkyl, (C₁₂-C₂₂)alkylacetate and hydroxyalkyl radicals; and aromatic radicals such as aryl and alkylaryl; and X⁻ is Chosen from halides, phosphates, acetates, lactates, (C₂-C₆) alkyl sulfates and alkyl- or alkylaryl-sulfonates;

quaternary ammonium salts of imidazoline;

diquaternary ammonium salts; and

quaternary ammonium salts comprising at least one ester function.

The quaternary ammonium salts mentioned above that may be Used in compositions according to the present invention include, but are not limited to tetraalkylammonium chlorides, for instance dialkyldimethylammonium and alkyltritnethylammonium chlorides in which the alkyl radical comprises from about 12 to 22 carbon atoms, such as behenyltrimethylammonium, distearyldimethylammonium, cetyltrimethylammonium and benzyldimethylstearylammonium chloride; palmitylamidopropyltrimethylammonium chloride; and stearamidopropyldimethyl(myristyl acetate)ammonium chloride, sold under the name “Ceraphyl® 70” by the company Van Dyk.

According to one embodiment, the cationic surfactant that may be used in the compositions of the present invention is chosen from quaternary ammonium salts, for example from behenyltrimethylammonium chloride, cetyltrimethylammonium chloride, Quaternium-83, Quaternium-87, Quaternium-22, behenylamidopropyl-2,3-dihydroxypropyldimethylammonium chloride, palmitylamidopropyltrimethylammoniurn chloride, and stearamidopropyldimethylamine.

The amount of the cationic surfactant(s) may range, for example, from 0.01 to 30% by weight, preferably from 0.05 to 20% by weight, and more preferably from 0.1 to 10% by weight, relative to the total weight of the composition.

(Amphoteric Surfactant)

The cosmetic composition according to the present invention may comprise at least one amphoteric surfactant, and two or more amphoteric surfactants may be used in combination. Thus, a single type of amphoteric surfactant or a combination of different types of amphoteric surfactants may be used.

The amphoteric surfactant is not limited. The amphoteric or zwitterionic surfactants can be, for example (nonlimiting list), amine derivatives such as aliphatic secondary or tertiary amine, and optionally quaternized amine derivatives, in which the aliphatic radical is a linear or branched chain comprising 8 to 22 carbon atoms and containing at least one water-solubilizing anionic group (for example, carboxylate, sulphonate, sulphate, phosphate or phosphonate).

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

R₁—CONHCH₂CH₂—N⁺(R₂)(R₃)(CH₂COO⁻)

in which:

R₁ denotes an alkyl radical of an acid R₁—COOH present in hydrolysed coconut oil, a heptyl, nonyl or undecyl radical,

R₂ denotes a beta-hydroxyethyl group, and

R₃ denotes a carboxymethyl group; and

R₁′—CONHCH₂CH₂—N(B)(C)

in which:

B represents —CH₂CH₂OX′,

C represents —(CH₂)_{z}—Y′, with z=1 or 2,

X′ denotes a —CH₂CH₂—COOH group, —CH₂—COOZ′, —CH₂CH₂—COOH, —CH₂CH₂—COOZ′ or a hydrogen atom,

Y′ denotes COOH, COOZ′, —CH₂—CHOH—SO₃Z′ or a —CH₂—CHOH—SO₃H radical,

Z′ represents an ion of an alkaline or alkaline earth metal such as sodium, an ammonium ion or an ion issued from an organic amine, and

R₁′ denotes an alkyl radical of an acid R₁′—COOH present in coconut oil or in hydrolysed linseed oil, an alkyl radical, such as a C₇, C₉, C₁₁ or C₁₃ alkyl radical, a C₁₇ alkyl radical and its iso form, or an unsaturated C₁₇ radical.

It is preferable that the amphoteric surfactant be selected from (C₈-C₂₄)-alkyl amphomonoacetates, (C₈-C₂₄)alkyl amphodiacetates, (C₈-C₂₄)alkyl amphomonopropionates, and (C₈-C₂₄)alkyl amphodipropionates

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C2M concentrate by the company Rhodia Chimie.

Use may also be made of the compounds of formula

Ra″—NH—CH(Y″)—(CH₂)ₙ—C(O)—NH—(CH₂)ₙ—N(Rd)(Re)

in which:
Ra″ represents a C₁₀-C₃₀ alkyl or alkenyl group of an acid Ra″—C(O)OH preferably present in hydrolyzed linseed oil or coconut oil;
Y″ represents the group C(O)OH, —C(O)OZ″, —CH₂—CH(OH)—SO₃H or the group —CH₂—CH(OH)—SO₃—Z″, with Z″ representing a cationic counter ion resulting from an alkali metal or alkaline earth metal, such as sodium, an ammonium ion or an ion resulting from an organic amine;
Rd and Re represent, independently of one another, a C₁-C₄ alkyl or hydroxyalkyl radical; and
n and n′ denote, independently of one another, an integer ranging from 1 to 3.

Mention may in particular be made of the compound classified in the CTFA dictionary under the name sodium diethylaminopropyl cocoaspartamide and sold by Chimex under the name Chimexane HB.

Preferably, the amphoteric surfactant may be a betaine.

The betaine-type amphoteric surfactant is preferably selected from the group consisting of alkylbetaines, alkylamidoalkylbetaines, alkylsulfobetaines, alkylphosphobetaines, and alkylamidoalkylsulfobetaines, in particular, (C₈-C₂₄)alkylbetaines, (C₈-C₂₄)alkylamido(C₁-C₈)alkylbetaines, (C₈-C₂₄)alkylsulphobetaines, and (C₈-C₂₄)alkylamido(C₁-C₈)alkylsulphobetaines. In one embodiment, the amphoteric surfactants of betaine type are chosen from (C₈-C₂₄)alkylbetaines, (C₈-C₂₄)alkylamido(C₁-C₈)alkylsulphobetaines, (C₈-C₂₄)alkylsulphobetaines, and alkyl(C₈-C₂₄)phosphobetaines.

Non-limiting examples that may be mentioned include the compounds classified in the CTFA dictionary, 9th edition, 2002, under the names cocobetaine, laurylbetaine, cetylbetaine, coco/oleamidopropylbetaine, cocamido propyl betaine, palmitamido propylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular cocobetaine and cocamidopropylbetaine.

It is more preferable that the amphoteric surfactant be selected from the group consisting of cocamidopropyl betaine, lauramidopropyl betaine, cocobetaine, lauryl betaine, cetyl dimethyl betaine, stearyl dimethyl betaine, cocodimethyl betaine, cocamidopropyl hydroxysulfate, disodium cocoamphodiacetate, sodium cocoamphoacetate, sodium lauriminodipropionate, lauryl hydroxysultaine, cocamidepropyl hydroxylsultaine, Coco alkyl dimethyl amine oxides, lauramine oxide and mixtures thereof.

The amount of the amphoteric surfactant(s) may range, for example, from 0.01 to 30% by weight, preferably from 0.05 to 20% by weight, and more preferably from 0.1 to 10% by weight, relative to the total weight of the composition.

The cosmetic composition according to the present invention may also comprise at least one water-soluble additive such as thickeners, sequestering agents, UV screening agents, preserving agents, vitamins or provitamins, opacifiers, fragrances, plant extracts, humectants, coloring materials, antioxidants, and proteins, provided that the cosmetic additive is in a free form. i.e., it is not included in the water-insoluble particle.

The cosmetic composition according to the present invention comprises at least 40% by weight of water.

The amount of water may be from 40 to 98% by weight or less, preferably from 45 to 95% by weight, more preferably from 50 to 90% by weight, and more preferably form 55 to 85% by weight, relative to the total weight of the composition.

The cosmetic composition according to the present invention can be prepared by mixing the above essential or optional components by using a conventional mixing means such as a mixer and a homogenizer.

The cosmetic composition according to the present invention can be used as a cosmetic product for a keratin substance such as skin or hair. The cosmetic product may be rinse-off or leave on products for hair (e.g., shampoos and conditioners), make-up removers for skin or mucous membrane (e.g., cleansing products), body wash products, and the like.

The cosmetic composition according to the present invention can improve the deposition of the water-insoluble particles on the skin or hair, and therefore, it can enhance the effects due to the water-insoluble particle, such as fragrance effects, to the skin or hair.

Particularly, the cosmetic composition according to the present invention can be a cleansing or washing composition for a keratin substance, preferably keratin fibers, and more preferably human hair. It could be a shampoo composition or a bath/shower composition.

[Cosmetic Process]

The cosmetic composition according to the present invention can be used in a cosmetic process for a keratin substance comprising the step of applying the cosmetic composition according to the present invention to a keratin substance.

The keratin substance here means a material containing keratin as a main constituent element, and examples thereof include skin, nails, lips, hair and the like.

The keratin substance can be in a dry state or in a wet state before application of the cosmetic composition according to the present invention. The application of the cosmetic composition according to the present invention to the keratin substance may or may not be followed by rinsing the keratin substance. Before rinsing, the cosmetic composition according to the present invention can be left in contact with the keratin substance, for example, from 30 seconds to 30 minutes.

It is preferable that the cosmetic composition according to the present invention be used in a process for cleansing a keratin substance, preferably keratin fibers, and more preferably human hair, wherein the cosmetic composition according to the present invention is applied to the keratin substance, preferably keratin fibers, and more preferably human hair. More preferably, the cosmetic composition according to the present invention will be rinsed off from the keratin substance.

The cosmetic composition according to the present invention can enhance the deposition of the water-insoluble particles on the keratin substance. Therefore, even if the cosmetic composition according to the present invention is washed away from the keratin substance, a relatively large amount of the water-insoluble particles can stay on the keratin substance. Thus, the keratin substance can enjoy benefits due to the water-insoluble particle, even after rinsing of the cosmetic composition according to the present invention. If the water-insoluble particle includes fragrance material(s), the keratin substance can enjoy the benefits caused by the fragrance material(s) such as stronger fragrance effects at desired times and prolonged fragrance effects.

### EXAMPLES

The present invention will be described in a more detailed manner by way of examples. However, these examples should not be construed as limiting the scope of the present invention.

### Examples 1-2 and Comparative Examples 1-4

### Preparation

The following shampoo compositions according to Examples 1-2 (Ex. 1-2) and Comparative Examples 1-4 (Comp. Ex. 1-4) were respectively prepared by mixing the ingredients shown in Table 1. The numerical values for the amounts of the ingredients are all based on “% by weight” as active raw materials.

TABLE 1

| | | | Comp. | Comp. | Comp. | Comp. |
|---|---|---|---|---|---|---|
| | Ex. 1 | Ex. 2 | Ex. 1 | Ex. 2 | Ex. 3 | Ex. 4 |
| Water | qs 100 | qs 100 | qs 100 | qs 100 | qs 100 | qs 100 |
| Sodium Laureth Sulfate | 15 | 15 | 15 | 15 | 15 | 15 |
| Coco-Betaine | 3 | 3 | 3 | 3 | 3 | 3 |
| Citric Acid | 0.08 | 0.1 | 0.08 | 0.1 | 0.1 | 0.1 |
| Sodium Benzoate | 0.5 | 0.5 | 0.5 | 0.5 | 0.5 | 0.5 |
| Salicylic Acid | 0.2 | 0.2 | 0.2 | 0.2 | 0.2 | 0.2 |
| Fragrance in Melamine | 0.8 | 0.8 | 0.8 | 0.8 | 0.8 | 0.8 |
| Microcapsule | | | | | | |
| Polyquaternium-67 | 0.5 | — | — | — | — | — |
| (SoftCAT ™ SX-1300X) | | | | | | |
| Polyquaternium-67 | — | 0.5 | — | — | — | — |
| (SoftCAT ™ SL-60) | | | | | | |
| Polyquaternium-10 | — | — | — | 0.5 | — | — |
| (JR-30M) | | | | | | |
| Guar | — | — | — | — | 0.5 | — |
| Hydroxypropyltrimonium | | | | | | |
| chloride | | | | | | |
| (Jaguar C13-S) | | | | | | |
| Polyquaternium-6 | — | — | — | — | — | 0.5 |
| (Merquat 100) | | | | | | |

[Olfactory Evaluation]

The olfactory evaluation of the shampoo compositions according to Examples 1-2 and Comparative Examples 1-4 was performed on hair, in accordance with the following protocol: Weigh 0.4 g of the shampoo composition per gram of a hair swatch (2.7 g/20 cm) in a watch glass. Moisten the hair swatch with tap water for five seconds while combing with the fingers. Squeeze the hair swatch between the fingers. Place the hair swatch in a watch glass and apply the shampoo composition along the hair swatch. Gently massage the shampoo composition into the hair swatch to lather the shampoo composition from the root to the tip six times for 15 seconds. Place the hair swatch on a clean watch glass and leave it for five minutes. Rinse the hair swatch with tap water until foam disappears. Comb the hair swatch and dry with dryer at 60° C. for 10 minutes for each gram of hair. Leave the hair swatch to cool down to ambient temperature.

The hair swatch was evaluated in terms of the fragrance intensity thereof, using a 0-10 scale (0: No odor, from 1 to less than 3: low odor, 3 to less than 5: medium odor, from 5 to less than 7: high odor, from 7 to less than 10: strong odor). The results of the evaluation were recorded as pre-comb fragrance intensity. Next, the hair swatch was combed three times, and was evaluated again in terms of the fragrance intensity thereof. The results of the evaluation were recorded as post-comb fragrance intensity. The results are shown in Table 2. Fragrance intensity with the scale of 3-10 can be clearly perceived.

TABLE 2

| | | Fragrance Intensity (0-10) | |
|---|---|---|---|
| | Cationic Polymer | Pre-Comb | Post-Comb |
| Ex. 1 | Polyquaternium-67 | 3 | 7 |
| Ex. 2 | Polyquaternium-67 | 2 | 4 |
| Comp. Ex. 1 | — | 2 | 2 |
| Comp. Ex. 2 | Polyquaternium-10 | 2 | 2.5 |
| Comp. Ex. 3 | Guar Hydroxypropyl- | 1.5 | 2 |
| | trimonium Chloride | | |
| Comp. Ex. 4 | Polyquaternium-6 | 2 | 2 |

The result indicates that cosmetic composition in the present invention can effectively deposit the microcapsule and deliver the stronger fragrance benefit to the hair.

### Example 3 and Comparative Examples 5-7

### Preparation

The following shampoo compositions according to Example 3 (Ex. 3) and Comparative Examples 5-7 (Comp. Ex. 5-7) were respectively prepared by mixing the ingredients shown in Table 3. The numerical values for the amounts of the ingredients are all based on “% by weight” as active raw materials.

TABLE 3

| | | Comp. | Comp. | Comp. |
|---|---|---|---|---|
| | Ex. 3 | Ex. 5 | Ex. 6 | Ex. 7 |
| Water | qs 100 | qs 100 | qs 100 | qs 100 |
| Sodium Laureth Sulfate | 15 | 15 | 15 | 15 |
| Coco-Betaine | 3 | 3 | 3 | 3 |
| Citric Acid | 0.08 | 0.08 | 0.08 | 0.08 |
| Sodium Benzoate | 0.5 | 0.5 | 0.5 | 0.5 |
| Salicylic Acid | 0.2 | 0.2 | 0.2 | 0.2 |
| Polyquaternium-67 | 0.5 | 0.5 | — | — |
| (SoftCAT ™ SX-1300X) | | | | |
| Guar Hydroxypropyl- | — | — | — | 0.5 |
| trimonium chloride | | | | |
| (Jaguar C13-S) | | | | |
| Particle | 1 | — | 1 | 1 |

The type of particle used is shown in Table 4 hereinafter.

[Particle Deposition Evaluation]

The particle deposition evaluation of the shampoo compositions according to Example 3 and Comparative Examples 5-7 was performed on hair, in accordance with the following protocol: Prepare the hair swatch treated with the shampoo composition in the same manner as in the above [Olfactory Evaluations].

The particle deposition on the surface of dried hair was observed by a digital microscope (AnMo Electronics Corp., Dino-Lite AM-413ZT, x 65). The results are shown in Table 4. In Table 4, “+” means that the particle was found on the hair fibers, and “−” means that the particle was not found on the hair fibers.

TABLE 4

| | | Comp. | Comp. | Comp. |
|---|---|---|---|---|
| Particle | Ex. 3 | Ex. 5 | Ex. 6 | Ex. 7 |
| Silicone-Treated Silica Beads | + | − | − | − |
| Micro-Porous Silica Particles | + | − | − | − |
| Cellulose Beads | + | − | − | − |
| Polyurea Powder | + | − | − | − |
| Lauryl Methacrylate/Glycol | + | − | − | − |
| Dimethacrylate Crosspolymer | | | | |
| Mica | + | − | − | − |
| Alumino-Silicate | + | − | − | − |
| Synthetic Fluorphlogopite | + | − | − | − |
| Talc | + | − | − | − |
| Vinyldimethicone/Methicone | + | − | − | − |
| Silsesquioxane Crosspolymer | | | | |
| Aerogel Beads | + | − | − | − |
| TiO₂ | + | − | − | − |

### Example 4-5 and Comparative Example 8-9

### Preparation

The following shampoo compositions according to Examples 4-5 (Ex. 4-5) and Comparative Examples 8-9 (Comp. Ex. 8-9) were respectively prepared by mixing the ingredients shown in Table 5. The numerical values for the amounts of the ingredients are all based on “% by weight” as active raw materials.

TABLE 5

| | | | Comp. | Comp. |
|---|---|---|---|---|
| | Ex. 4 | Ex. 5 | Ex. 8 | Ex. 9 |
| Water | qs 100 | qs 100 | qs 100 | qs 100 |
| Sodium Laureth Sulfate | 15 | 15 | 15 | 15 |
| Coco-Betaine | 3 | 3 | 3 | 3 |
| Citric Acid | 0.08 | 0.08 | 0.08 | 0.08 |
| Sodium Benzoate | 0.5 | 0.5 | 0.5 | 0.5 |
| Salicylic Acid | 0.2 | 0.2 | 0.2 | 0.2 |
| Polyquaternium-67 | 0.5 | 0.5 | 0.5 | 0.5 |
| (SoftCAT ™ SX-1300X) | | | | |
| Aerogel Beads | 0.5 | — | — | — |
| Fragrance | 1 | — | — | — |
| Lauryl Methacrylate/Glycol | — | 0.5 | — | — |
| Dimethacrylate Crosspolymer | | | | |
| Fragrance | — | 1 | — | — |
| Fragrance | — | — | 1 | — |

In Examples 4-5, fragrance-impregnated particles were used. In Example 4, aerogel beads absorbing fragrance were used. In Example 5, lauryl methacrylate/glycol dimethacrylate crosspolymer particle absorbing fragrance was used. In Comparative Example 8, free fragrance was used.

[Particle Deposition Evaluation]

The particle deposition on the surface of dried hair was observed in the same manner as explained above. The results are shown in Table 6. In Table 6, “+” means that the particle was found on the hair fibers, and “−” means that the particle was not found on the hair fibers.

[Olfactory Evaluation]

The fragrance duration on hair was evaluated by an olfactory evaluation as described hereinbefore in comparison with Comparative Example 8 (1: much shorter than Comparative Example 8, 2: shorter than Comparative Example 8, 3: almost the same as Comparative Example 8, 4: longer than Comparative Example 8, 5: much longer than Comparative Example 8). The results are shown in Table 6.

TABLE 6

| | | | Comp. |
|---|---|---|---|
| | Ex. 4 | Ex. 5 | Ex. 8 |
| Particle Deposition | + | + | − |
| Fragrance Duration | 5 | 4 | − |

[Headspace Analysis]

The collection of volatile fragrance molecules released from hair was performed by a dynamic headspace. Nitrogen gas was passed through the collecting apparatus with 100 mL/min for 30 minutes at 40° C. and absorbed onto TENAX TA®. The fragrance quantity in nitrogen was estimated by thermic desorption of the TENAX TA® on Gestel TDS3 system and analysis by GC/MS (Agilent technologies 7890A/5975C). Linalool was monitored as a representative of volatile fragrance component. The results are shown in Table 8, as a relative area count relative to the results for Comparative Example 8.

TABLE 7

| | | | Comp. |
|---|---|---|---|
| | Ex. 4 | Ex. 5 | Ex. 8 |
| Relative Area Count | 325 | 173 | 100 |

In view of the experimental results shown above, it is clear that the present invention can effectively deposit a fragrance or particles on the hair, and therefore, the present invention can provide cosmetic effects based on the fragrance or particles in a stronger manner and/or for a longer period of time.

In case of using Aerogel beads as the particle (Example 4), the amount of particles deposited on the hair fibers was determined by elemental analysis for silicon (Si) using inductively-coupled plasma optical emission spectrometry (ICP-OES). The results are shown in Table 8. For Comparative Examples 8 and 9, the same analysis was made in order to determine the amount of silicon when Aerogel particle is not used.

TABLE 8

| | | Comp. | Comp. |
|---|---|---|---|
| | Ex. 4 | Ex. 8 | Ex. 9 |
| Si Amount (mg/kg) | 210 | 90 | 50 |

1. A cosmetic composition, comprising:
(a) at least one anionic surfactant;
(b) at least one hydrophobically-modified cationic polymer;
(c) at least one water-insoluble particle comprising at least an inorganic part and/or a polymeric part and/or a visible light absorbing part; and
(d) at least 40% by weight of water.

2. The cosmetic composition according to claim 1, wherein the amount of the (b) hydrophobically-modified cationic polymer is 0.005% by weight or more, preferably 0.01% by weight or more, and more preferably 0.05% by weight or more, relative to the total weight of the composition.

3. The cosmetic composition according to claim 1 or 2, wherein the amount of the (b) hydrophobically-modified cationic polymer ranges from 0.005% to 10% by weight, preferably from 0.01% to 5% by weight, and more preferably from 0.05% to 1% by weight, relative to the total weight of the composition.

4. The cosmetic composition according to any one of claims 1 to 3, wherein the amount of the (a) anionic surfactant ranges from 1 to 30% by weight, preferably from 3 to 25% by weight, and more preferably from 5 to 20% by weight, relative to the total weight of the composition.

5. The cosmetic composition according to any one of claims 1 to 4, wherein the (b) hydrophobically-modified cationic polymer is selected from the group consisting of
(1) cationic associative amphiphilic polyurethanes,
(2) quaternized cellulose derivatives,
(3) cationic poly(vinyllactam) polymers, and
(4) cationic polymer(s) obtained by polymerization of a monomer mixture comprising one or more vinyl monomers substituted with one or more amino groups, one or more hydrophobic nonionic vinyl monomers, and one or more associative vinyl monomers.

6. The cosmetic composition according to claims 1 to 5, wherein the (b) hydrophobically-modified cationic polymer comprises at least one quaternary ammonium group.

7. The cosmetic composition according to claim 6, wherein the quaternary ammonium group includes at least one C₃-C₃₀ hydrocarbon group.

8. The cosmetic composition according to claims 1 to 7, wherein the (b) hydrophobically-modified cationic polymer is a cellulose compound.

9. The cosmetic composition according to any one of claims 1 to 8, wherein the (c) water-insoluble particle is in the form of a microcapsule or a microsphere.

10. The cosmetic composition according to any one of claims 1 to 9, wherein the (c) water-insoluble particle comprises at least one inorganic material selected from the group consisting of talc, mica, silica, kaolin, sericite, calcinated talc, calcinated mica, calcinated sericite, synthetic mica, bismuth oxychloride, barium sulfate, boron nitride, calcium carbonate, magnesium carbonate, magnesium hydrogen carbonate, and hydroxyapatite.

11. The cosmetic composition according to any one of claims 1 to 9, wherein the (c) water-insoluble particle comprises at least one organic material selected from the group consisting of polyurea, melamine-formaldehyde condensate, urea-formaldehyde condensate, aminoplasts, polyurethane, polyacrylate, polyphosphate, polystyrene, polyester, polyamide, polyolefin, polysaccharide, silicone, silicone resin, protein, modified cellulose, and gum.

12. The cosmetic composition according to any one of claims 1 to 11, wherein the (c) water-insoluble particle includes at least one additional cosmetic active agent, preferably hydrophobic and/or lipophilic cosmetic active agent, and more preferably a fragrance, a conditioning agent or a UV filter.

13. The cosmetic composition according to any one of claims 1 to 12, wherein the amount of the (c) water-insoluble particle ranges from 0.01 to 10% by weight, preferably from 0.05 to 5% by weight, and more preferably from 0.1 to 3% by weight, relative to the total weight of the composition.

14. The cosmetic composition according to any one of claims 1 to 13, wherein the (a) anionic surfactant is be selected from the group consisting of:
sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C14-16 olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, disodium cocoyl glutamate, potassium myristoyl glutamate, TEA-cocoyl glutamate, sodium cocoyl glycinate, potassium cocoyl glycinate, sodium cocoyl alaniate, TEA-cocoyl alaninate and mixtures thereof.

15. The cosmetic composition according to any one of claims 1 to 14, further comprising at least one amphoteric or cationic surfactant.

16. The cosmetic composition according to any one of claims 1 to 15, wherein the amount of (d) water is from 40 to 98% by weight or less, preferably from 45 to 95% by weight, and more preferably from 50 to 90% by weight relative to the total weight of the composition.

17. A process for cleansing a keratin substance, preferably keratin fibers, and more preferably human hair, wherein the cosmetic composition according to any one of claims 1 to 16 is applied to the keratin substance, preferably keratin fibers, and more preferably human hair.
